(19) United States
(12) Patent Application Publication
(10) Pub. No.: US 2007/0088171 A1
(43) Pub. Date: Apr. 19, 2007

(54) Selective hydrogenation of functional groups in substrates and partially hydrogenated fatty acids and fatty acid derivatives

(76) Inventors: Magnus Harrod, Alingsas (SE); Andre Holmqvist, Goteborg (SE); Sander Van Den Hark, Goteborg (SE)

Correspondence Address: YOUNG & THOMPSON, 745 SOUTH 23RD STREET, 2ND FLOOR, ARLINGTON, VA, 22202, US

(21) Appl. No.: 11/529,761

(22) Filed: Sep. 29, 2006

Related U.S. Application Data
(63) Continuation of application No. PCT/SE05/00481, filed on Mar. 31, 2004.

(30) Foreign Application Priority Data
Mar. 31, 2004 (SE) 0400868-6

(51) Int. Cl. C07C 51/36

(52) U.S. Cl. 554/145

(57) ABSTRACT

Process for the hydrogenation of functional groups in hydrogenatable substrates, wherein hydrogen gas is mixed with the substrate and a solvent, the mixture is brought into contact with a catalyst. The hydrogenation is carried out under process conditions which are adapted to the activity of the catalyst used, wherein the temperature is sufficiently low, the substrate concentration is sufficiently high and the diffusivity is sufficiently high to provide a selective hydrogenation of the functional group having a higher reactivity than one which has a lower reactivity. The functional groups can for example comprise different C═C-groups in substrates in the form of lipids, primarily fatty acids and fatty acid derivatives, such as triglycerides and methyl fatty acids. Partially-hydrogenated fatty acids/fatty acid derivatives can hereby be obtained, which possess a low content of trans-fatty acid content as well as high selectivity in the form of S_{Ln}-number and S_{Lo}-number.

### TECHNICAL FIELD

The present invention concerns a process for the hydrogenation of functional groups in hydrogenatable substrates especially in the form of lipids, primarily fatty acids and fatty acid derivatives, e.g. triglycerides and methyl fatty acids, wherein hydrogen gas is mixed with a solvent and the substrate in the presence of a catalyst, and the reaction is carried out under predetermined conditions of pressure, time, temperature and concentration, so that the reaction reaches maximum selectivity. Selectivity is obtained under substantially homogeneous conditions by reaching a very favourable balance between mass transport (addition of substrate and removal of product) and reaction rate. To provide this, supercritical solvents are especially suitable.

By this technique, one can control the reactions so that one obtains completely unique selectivities. In the case of fatty acids, we can, for example, selectively hydrogenate fatty acids with 3 double bonds, with reduced formation of trans- and no formation of saturated fatty acids. Another example is to hydrogenate fatty acids with 2 and 3 double bonds without hydrogenating monounsaturated fatty acids and simultaneously obtain very low amounts of trans-fatty acids. The invention also concerns partially hydrogenated fatty acids and fatty acid derivatives produced according to this process.

### BACKGROUND TO THE INVENTION

Catalysis

The reaction rate of various catalytic reactions depends on the type of catalyst, temperature, concentrations (substrate, products), time, adsorption coefficients and equilibrium constants. Transport mechanisms between bulk and catalysts are often important for the result of the reaction (Moulijn et al. 1993). One can control the reaction by influencing these fundamental factors in different ways.

We will describe how one can control the reaction in a new way, and obtain unique selectivities for different hydrogenation reactions by choosing suitable solvents, suitable catalysts, suitable temperatures, suitable times, suitable total pressures and suitable concentrations of both hydrogen and substrate on the surface of the catalyst. Unique product qualities have hereby been obtained.

Selectivity

The concept of selectivity can be defined in many different ways. In this document, we divide selectivity into four levels:
1. Selectivity between different functional groups, e.g. between hydrogenation of C═C and C═O. This is the most basic selectivity.
2. Selectivity between the same functional group in a reaction mixture of different molecules, e.g. between different C═C groups in a reaction mixture of fatty acids with different C═C groups.
3. Selectivity between the same functional group in the same molecule, e.g. between different C═C groups in a fatty acid or a triglyceride.
4. Selectivity between different chiral isomers.

Selectivity between different functional groups is usually not difficult to obtain, e.g. by choosing suitable catalysts or by choosing suitable temperature ranges.

Selectivity between the same functional group is significantly harder. In this patent, we illustrate methods for how this might be achieved. The difference in absorption coefficients and reaction rates for the various C═C bonds in fatty acids is very small. By showing that we can obtain good selectivity in this system, we have shown that the principles generally apply for the selectivities 1, 2 and 3 as described above.

Chiral selectivity requires chiral catalysts. The principles which we use in this patent may also be used in such reactions, but this has not been investigated.

Functional Groups

Our principles can be suited to all functional groups which can be hydrogenated. Some examples of functional groups which can be hydrogenated are C═C, C═O, COOR, CN, NO₂, aromatic, etc. It can also be decomposition with hydrogen, e.g. removal of sulfur, oxygen, nitrogen, or to open cyclic rings.

We demonstrate the principles by hydrogenation of C═C bonds in fatty acids, methyl fatty acids, ethyl fatty acids and triglycerides. Triglycerides are very large (MW ca. 900) non-polar molecules. The diffusivity decreases with increasing molecular weight. This means that the mass transport of triglycerides becomes low, and it is extra hard to obtain selective hydrogenation of the different fatty acids in the molecule. Together, our molecules cover a very large range of molecules.

Hydrogenation of Fatty Acids, Methyl Fatty Acids, Triglycerides and Other Fatty Acid Derivatives.

Approximately 90 million tonnes of vegetable oil is produced every year (Mielke 1992), of which ca. 20% is hardened (hydrogenated). Around 2 million tonnes of marine oil is hydrogenated every year. Production is spread over the entire industrialised world. Through hydrogenation, hydrogen is added to the double bonds of the unsaturated fatty acids. A large portion of the oils are only partially hydrogenated. In this way, the desired melting properties and the desired consistency of the fats is obtained, primarily for production of margarine and shortening. Furthermore, the oxidation resistance is increased by hydrogenation, and through this the lifetime of the fats is increased (Swern 1982).

Among other things, a problem with the hydrogenation processes of today is that new fatty acids which do not occur in nature are produced to a large extent. These are often designated in combination with trans-fatty acids, but the double bonds change both position and form (cis-trans) under hydrogenation (Allen 1956, Allen 1986).

From a technical and functional point of view, trans-fatty are usually desirable (Swern 1982). The health effects of trans-fatty acids are being questioned to a very rapidly expanding extent (Wahle & James 1993).

In 2003, this led to Denmark introducing legislation on the maximum amount of trans-fatty acids in food products. In the USA, legislation will be introduced on marking the amount of trans-fatty acids in food products on 1 Jan. 2006.

The rather complicated reaction scheme which can occur upon hydrogenation of polyunsaturated to saturated fatty acids in triglycerides can be described in a simple way by equation (1) 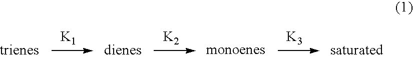
in which K₁, K₂, K₃ are reaction rate constants in the process. The literature provides the equations which are needed to calculate these constants from the concentrations of the various fatty acids (Hui, 1996). One tends to describe the selectivity between these reactions with S_{Ln} and S_{Lo}, which are defined in eq. 2 and 3. When these parameters are larger than 1, one begins to indicate selective processes (Hui, 1996)
S_{Ln}=K₁/K₂ (2)
S_{Lo}=K₂/K₃ (3)

Note that all reactions leading to production of trans-fatty acids and all isomerisation reactions are ignored in the above reaction scheme; one simply counts the number of double bonds in the fatty acids. The common understanding is that the probability of a C═C bond reacting is determined by “simple arithmetic probability” [Hui, 1996, p 218]. However, there are researchers who consider that double bonds furthest from the carbonyl group have the highest reactivity. (Hsu, 1989).

Production of trans-fatty acids is often described by another index, S_{II} which is defined with eq. 4 wherein IV is the iodine number.
S_{II}=% trans/(change in IV) (4)

A typical traditional hydrogenation reactor is a large tank (5-20 m³) filled with oil and hydrogen, together with finely-divided catalyst (nickel metal powder). The reaction is carried out at low pressure, just above atmospheric pressure (0.5-5 bar), and high temperature (130-210° C.). Much care is taken in mixing the hydrogen in the oil, as this is a factor which limits the reaction rate (Grau et al., 1988). When the oil is half-hydrogenated, the amount of trans-fatty acids is normally between 30 and 50%.

According to the “half hydrogenation” theory, the concentration of the activated H-atoms on the catalyst surface determines how much of the double bounds are hydrogenated or deactivated without hydrogenating. A deficiency of activated H-atoms causes trans- and positional isomerisation (Allen, 1956; Allen, 1986). Deficiency of activated H-atoms arises through low solubility of H₂ in the oil. The “half hydrogenation” theory and empirical results thus agree very well (Allen, 1956; Allen, 1986; Hsu et al., 1989).

Comprehensive literature exists on the hydrogenation of food oils. The lowest amount of trans-fatty acids is obtained with high hydrogen pressure and low temperature. If the H₂ pressure is raised (from 3 to 50 bar) during partial hydrogenation of soya oil (iodine number start=135, end=70), the amount of trans is reduced (from 40 to 15%, S_{II} from 61 to 23). Positional isomerisation is reduced to a corresponding degree. (Hsu et al., 1989). Commercially, these results are not sufficient. Legislation requires much lower amounts of trans-fatty acids. From a functional point of view, one must achieve a very low rate of formation of trans-fatty acids and simultaneously a high selectivity.

By adding a solvent which dissolves both the oil and the hydrogen, a significantly homogeneous phase can be produced. In this way, one can control the amount of hydrogen on the catalyst and achieve both enormous reaction rates and avoid production of trans-fatty acids in solid-bed reactors (WO9601304, Härröd, Möller, 1996; U.S. Pat. No. 6,265,596, Härröd, Möller; Härröd et al, 2001). Selectivity is practically absent, and one has not succeeded in preventing the accumulation of saturated fatty acids (Macher, 2001) in these processes.

There are many articles in the literature which describe how the selectivity increases by increasing the proportion of trans-fatty acid in the product. This type of selectivity is not considered in this review.

Copper is a catalyst which can selectively hydrogenate polyunsaturated fatty acids or fatty esters to monounsaturated fatty acids. It gives almost no saturated fatty acids, but it provides a lot of trans-fatty acids.

In an early patent, GB670906 (Miyake, 1952), it is described how a copper-chrome catalyst, free from activators, selectively hydrogenates polyunsaturated oils, fats or esters to monounsaturated, but that monounsaturated are not further hydrogenated to saturated (100-230° C., 1-80 bar H₂). In this patent, activators are defined as more active metals, such as Ni, Co, Pd, Pt. No ratio between cis/trans is given.

Unilever have shown that copper is a good catalyst for selective hydrogenation of soya oil. They show that the shelf-life of soya oil correlates very strongly with the amount to linolenic acid (18:3) in the oil. Using copper, one could obtain as low as 0.5% w/w 18:3 with an IV of 112 and a trans amount of 13% w/w (185° C., 5 bar H₂). Using nickel, as low as 0.5% w/w 18:3 with an IV of 91 and a trans amount of 25% w/w (185° C., 1 bar H2) could be obtained. (Okkerse et al 1967)

Cu/Zn is a catalyst which is even more selective for monounsaturated fatty acids, DE 4103 490 (Gritz, Göbel, 1992). The elaidic acid/stearic acid ratio is very high (0.82) (100-250° C., 1-50 bar H₂).

WO03059505 (Sleeter, 2003) describes copper-chrome as a good katalyst for selective hydrogenation of C═C in polyunsaturated oils. The amount of monounsaturated fatty acid is very high (ca. 80%), while the amount of trans is also very high (ca. 40%) (200-300° C., 20-35 bar H₂, 3-8 h).

In U.S. Pat. No. 4,278,609 (Kuiper, 1981) it is claimed that addition of ammonia improves selectivity for Pd, Pt, Ro and Ir. [−20-100° C. in claims (25° C. in most examples), <10 bar H₂, 75% solvent (acetone, dimethylformamide, ethylacetate, isopropanol, hexane, diglyme, tetrahydrofuran), 25% oil]. The data which is presented describes the selectivity at a low degree of hydrogenation. The starting material, soya oil, is hydrogenated so that C18:3 is reduced from 7 to 2%. Upon addition of ammonia, 7% trans-fatty acid is produced, 2.6% 18:1cis, but no saturated fatty acids are produced; without addition of ammonia 12% trans-, 11.2% 18:1 and 1.4% saturated fatty acids (Kuiper, 1981, Table I) is produced. With increasing amount of ammonia, the hydrogenation rate for 18:2 decreases (Kuiper, 1981, Tabell I och III)

Similar results have also been achieved with addition of diamines (U.S. Pat. No. 4,307,026, Kuiper, 1981).

### OBJECT AND SUMMARY OF THE INVENTION

Our goals of this invention are to be able to control the process such that unique selectivities can be obtained. A few examples are:
1. The majority of ccc-fatty acids in triglycerides are hydrogenated to cc-fatty acids without a significant portion of other C═C bonds being hydrogenated or being converted to trans- or isomerising.
2. The majority of ccc and cc-fatty acids are hydrogenated to c-fatty acids without a significant portion of other C═C bonds being hydrogenated or converted to trans- or isomerising.
3. The majority of c-fatty acids in ethyl fatty acids are hydrogenated without a significant portion of the existing trans-fatty acids being hydrogenated.
4. Conjugated fatty acids are hydrogenated to trans-fatty acids without formation of any saturated fatty acids.

These examples illustrate that we obtain unique selectivities with our methods.

According to the invention, this has been achieved by a process in which hydrogen gas is mixed with the substrate and a solvent, the mixture is brought into contact with a catalyst under predetermined conditions of concentration, pressure, temperature and time, and that hydrogenation is carried out under process conditions which are adapted to the activity of the catalyst used, wherein the temperature is sufficiently low, the substrate concentration is sufficiently high and the diffusivity is sufficiently high to provide a selective hydrogenation of a functional group having a higher reactivity than one which has a lower reactivity.

The mixture of hydrogen, substrate and solvent preferably forms a substantially homogeneous phase which is brought into contact with the catalyst.

According to one embodiment of the invention, the substantially homogeneous mixture of hydrogen, substrate, product and solvent is in a near-critical or critical state.

Preferably, the functional group which shows the lower reactivity first begins to be hydrogenated when at least 75%, preferably at least 80% and most preferably at least 90% of the functional group which shows the higher reactivity has been hydrogenated.

The hydrogenatable functional groups are—according to one aspect of the invention—of the same type but show different reactivities.

Alternatively, the hydrogenatable functional groups are of the same type but show different, yet similar, reactivities.

According to one aspect of the invention, the substrate is a mixture of different molecules and that the hydrogenatable functional groups of the same of different type occur in different molecules.

Alternatively, the hydrogenatable functional groups of the same or different type occur in the same molecule.

According to one embodiment of the invention, the hydrogenatable functional groups are C═C groups.

The substrate according to one aspect of the invention is lipids, primarily fatty acids and fatty acid derivatives, such as triglycerides and methyl fatty acids.

According to a further aspect of the invention, the substrate is fatty acids and/or fatty acid derivatives which are hydrogenated to a degree of reduction of 18:3, i.e. ccc-fatty acids of at least 85%, characterised in that the process conditions are adapted so that the hydrogenated final product comprises an amount of trans-fatty acids in the form of S_{II}-number of highest 50, and a selectivity in the form of S_{Ln}-number of at least 1.5.

The solvent is suitable selected so that it can dissolve at least 2% w/w, preferably at least 5% w/w and most preferably at least 10% w/w of the substrate at the process conditions in question.

For lipid substrates, the solvent is suitably selected from the group: carbon dioxide, ethane, propane, butane, pentane, hexane, heptane, tetrahydrofuran (THF), dioxane, dimethylether (DME), methanol, ethanol, acetone and mixtures thereof.

Preferably, for lipid substrates, the solvent is selected from the group: propane, butane, pentane, hexane, heptane, dimethylether (DME), ethanol, acetone and mixtures thereof. Most preferably, for lipid substrates, the solvent is selected from the group: propane, butane, dimethylether (DME), ethanol, acetone and mixtures thereof.

For triglyceride substrates, the solvent is selected from the group: butane and dimethylether (DME).

Preferably, the reaction is carried out in the absence of ammonia and diamines.

According to one aspect of the invention, the concentration of the substrate is at least 2% w/w, preferably at least 5 and most preferably at least 10% w/w.

Those catalysts which are used in the process of the invention are suitably solid-bed catalysts.

The reaction temperature of the process is suitably at most 200° C., preferably at most 100° C., and most preferably at most 75° C.

The hydrogen pressure in the substantially homogeneous mixture should suitably be at least 1 bar, preferably at least 2 bar and most preferably at least 5 bar.

The reaction time is suitably at least 1 sec, preferably at least 2 sec and most preferably at least 5 sec.

Upon hydrogenation of fatty acids and fatty acid derivatives, primarily triglycerides, the hydrogenation activity should be at the most 2.5 mmol H₂/l·s, preferably at most 1.5 mmol H₂/l·s and most preferably at most 1 mmol H₂/l·s.

Upon hydrogenation of fatty acids and fatty acid derivatives, primarily triglycerides, the hydrogenation activity should be at least 0.05 mmol H₂/l·s.

According to one embodiment the hydrogenation reaction is carried out in sequential steps, so that the majority of a certain type of a selected functional group with a certain reactivity is selectively hydrogenated in each step.

The different sequential hydrogenation steps are—according to one embodiment—carried out in different reactors with different reaction conditions with regard to one or more factors such as catalyst, temperature or pressure.

The invention concerns a partially-hydrogenated product in the form of fatty acids or fatty acid derivatives which have a reduction level of 18:3, i.e. ccc-fatty acids, of at least 85% characterized in that it has a trans-fatty acid content in the form of S_{II}-number of at most 50, and a selectivity in the form of S_{Ln}-number of at least 1.5.

The invention further concerns a partially-hydrogenated product in the form of fatty acids or fatty acid derivatives which have a reduction level of 18:3, i.e. ccc-fatty acids, under 100%, characterized in that it has a trans-fatty acid content in the form of S_{II}-number of at most 40, preferably at most 30 and a selectivity in the form of S_{Ln}-number of at least 3.5, preferably at least 4.

The invention further concerns a partially-hydrogenated product in the form of fatty acids or fatty acid derivatives which have a reduction level of 18:2+, i.e. ccc+cc fatty acids, of at least 35%, characterized in that it has a content of trans-fatty acids in the form of S_{II}-number of at most 50, and a selectivity in the form of S_{Lo}-number of at least 3.5.

In addition, a partially-hydrogenated product is considered, characterized in that it has a reduction level of 18:2+, i.e. ccc+cc fatty acids, of at least 20%, an amount of trans-fatty acids in the form of S_{II}-number of at most 40, preferably at most 30 and a selectivity in the form of S_{Lo}-number of at least 4, preferably at least 8.

Fundamental Idea

The key to good selectivity is that the mass transport to and from the catalyst is sufficiently large that it does not limit the reaction rate. In this invention:
1. we maximise the mass transport by adding a solvent with high diffusivity, low viscosity and high solvating ability of the substrate
2. we select a solvent with a high hydrogen solubility, so that we can attain substantially homogeneous conditions. In this way, we minimise isomerisation reactions.
3. we minimise the reaction rate by lowering the temperature and by choosing catalysts with low activities.

In this way, we have obtained unique product qualities. The principles are general, and apply to all hydrogenation reactions.

The catalysts react primarily with the bond which reacts most easily. When this bond cannot be transported sufficiently quickly to the catalyst surface, the catalyst takes the next most reactive bond. In this way, selectivity is worsened. The concentration of that functional group which is to be hydrogenated on the catalyst surface should be controlled so that the selectivity is maximised and not limited by mass transport of this group.

In order to maximise the above selectivity, the mass transport of the substrate and product must be so large that it does not limit the reaction rate; i.e. we must both provide the catalysts with reagents (both substrate with the correct type of binding and hydrogen) and transport the product away from the catalyst so rapidly that the next bond does not have a chance to react.
All measures which increase mass transport are good for selectivity
All measures which minimize the reaction rate are good for selectivity

Reaction rates tend to be more temperature-dependent that transport rates. This is a further reason for minimizing the temperature in order to maximize the desired selectivity. If the activation energy for hydrogenation of the various bonds is favourable, lowering the temperature can increase the selectivity further, and vice versa.

One disadvantage of lower temperature is that the entire reaction rate is reduced, and this leads to the reactor volume being larger.

Realization of the Fundamental Idea

Balance Between Transport Rate and Reaction Rate

For high selectivity, it is crucial that the transport rate is sufficiently high that it does not limit the reaction rate.

Transport Rates

The transport rate is determined by the product of diffusivity and the difference in concentration between bulk and the catalyst surface. In this invention, we control the concentration of the substrate on the catalyst surface, i.e. that bond which we wish to react, partly through bulk concentration and partly through choosing a solvent with good transport properties, i.e. high diffusivity and low viscosity and partly through reactor design, fixed bed reactor and small catalyst particles.

The amount of hydrogen must be high enough to minimise production of trans-bonds. To succeed in this, we preferably choose a solvent which gives a “substantially homogeneous” mixture. This term is defined below.

Reaction Rate

The reaction rate may be controlled by the choice of catalyst, temperature, substrate concentration and amount of hydrogen on the catalyst.

Each catalyst has its own optimal temperature for a given reaction. A more active catalyst requires a lower temperature to reach the same activity. One usually ranks the normal metals which hydrogenate C═C bonds according to: Cu<Ni<Pt<Pd.

With different support material, pore structures, metal concentrations and different additives, one can modify catalyst activity in different ways. One can, for instance, lower the concentration of Pd and obtain the same reactivity per unit volume with this catalyst as for a Cu-catalyst with the high concentration.

Hydrogenation reactions are—as a rule—strongly exothermic. Heat is released, and the temperature of the catalytic site rises sharply.

When one has a substrate such as triglycerides in which each molecule comprises a mixture of ccc-, cc-, c- and saturated fatty acids, it is important to control the reaction rate very carefully. If the reaction rate of ccc becomes so high that the temperature at some point in the reactor increases so much that cc begins to react to a substantial extent, there is a great risk that the temperature increases much further so that even c is hydrogenated to a substantial extent (see FIG. 5). If we attempt to limit the turnover by access to hydrogen, a lot of trans and other isomers are formed.

To control selectivity, it is important to control the temperature of the catalyst surface. Significant parameters for this are: the catalyst activity per unit volume; uniform activity in the entire reactor (no local high catalyst activities); temperature in the bulk flow; thermal diffusivity in the reaction mixture; hydrogen and substrate concentrations on the catalyst; and a perfect flow pattern in the reactor so that no hot points are formed in the reactor.

High amounts of hydrogen on the catalyst provides high reaction rates, which in turn leads to high transport requirements of the oil with the correct type of bonding to the catalyst.

To succeed in achieving the desired selectivity, it is important to investigate reaction rates and selectivities at a low a temperature as possible, where the catalyst has a low, yet evenly-spread activity.

A solvent which creates a substantially homogeneous phase with good thermal diffusivity, good mass diffusivity and a catalyst with a uniform and low activity are crucial to obtain perfect results.

Substantially Homogeneous

The definition substantially homogeneous means that the majority of the hydrogen exists in the continuous phase which covers the surface of the catalyst. The majority of the hydrogen should in this patent be interpreted such that sufficient hydrogen exists in the phase which covers the surface of the catalyst, so that there is not a shortage of hydrogen on the surface of the catalyst.

One way of checking that one has a substantially homogeneous mixture is to observe the reaction rate, which increases dramatically when the continuous phase covering the catalyst surface is substantially homogeneous. See also the description of FIGS. 1-6 below.

Substrate

We illustrate the method in detail with selective hydrogenation of C═C bonds in triglycerides, ethyl fatty acids and fatty acids, as well as selective hydrogenation of ester groups in malic acid dimethyl ester. In selective hydrogenation of fatty acids, it is important to solve the challenge of simultaneously attaining selective hydrogenation of the various C═C bonds and at the same time minimise formation of trans-fatty acids. Trans-bonds occur in parallel reactions to hydrogenation (see FIG. 7). To minimise formation of trans-bonds, we must carry out the reactions with a high concentration of hydrogen on the catalyst.

At the same time, higher amounts of hydrogen give higher reaction rates, which in turn lead to higher transport requirements of oil with the correct type of bond to the catalyst. There is therefore a conflict between minimising formation of trans-fatty acids and maximising the selectivity between ccc-, cc-, and c-fatty acids.

### BRIEF DESCRIPTION OF THE FIGURES

In the following, we will describe in principle what we mean with “substantially homogeneous” with various phase diagrams, with phase boundaries as a function of composition with constant temperature and pressure (FIGS. 1-4) as well as with phase boundaries as a function of temperature and pressure with constant composition (FIG. 5). FIG. 6 shows how one can detect where the boundary lies between substantially homogeneous and traditional reaction mixtures through a series of experiments.

FIG. 7 and FIG. 8 describe reaction pathways upon hydrogenation of C═C bonds in fatty acids and fatty acid derivatives.

### DESCRIPTION OF REACTION CONDITIONS WITH FIGURES

Supercritical solvents such as butane and DME are the two most interesting solvents for hydrogenation of triglycerides. We do not know of any calculated phase diagram for this system. In order to describe the principles for substantial homogeneity at various process conditions, we have assumed that the curves resemble FIG. 1 at 180 bar and 130° C. The Figure shows calculated data for the propane, hydrogen and soya bean oil system, at the given pressures and temperatures (Weidner et al. 2002). The scales on the axes are mol % and go from 0 to 100% on all axes. The darker area indicates homogeneous mixtures, and the lighter area indicates which mixtures give rise to two phases. This figure is also used in FIGS. 2-4.

Hydrogen consumption is a very important parameter for how the mixtures reacts in a solid-bed reactor. We have therefore defined the hydrogen requirement as the amount of hydrogen which is consumed (mol_{hydrogen}/mol_{substrate}) during one run of the reactor at the current process conditions (solvent, total pressure, catalyst, temperature, hydrogen pressure, substrate concentration, reaction time).

FIG. 2 illustrates the substantially homogeneous region in which the hydrogen requirement is maximal. All double bonds can be hydrogenated to saturated bonds at the given process conditions. The dashed line between “Need” and “Butane” shows where the stoichiometric limit lies. To the right of the line, an excess of hydrogen is prevalent. In the smaller portion of the dark area lying to the right of the dashed line, stoichiometric excess and single-phase conditions (c.f. with FIG. 1) prevail. In the slightly larger white area to the right of the dashed line, substantially homogeneous conditions prevail. Through a series of measurements of the reaction rate at various concentrations, one can determine both where single-phase conditions occur and above all, where one has substantially homogeneous conditions (see FIG. 6).

In FIG. 3, the hydrogen requirement is low due to the process conditions selected. Under these conditions, all ccc and all cc is hydrogenated to c. The reaction stops at the chosen conditions, before any saturated fatty acids are formed (ccc signifies fatty acids with 3 C═C and cc fatty acids with 2 C═C). We see that the substantially homogeneous area has become slightly larger, and it is the upper right area where one can avoid hydrogen shortage on the catalyst.

FIG. 4 illustrates a situation in which the hydrogen requirement is very low due to the selected process conditions. It is only ccc-fatty acids which are hydrogenated to cc-fatty acids. At this very low hydrogen requirement, mass transport of hydrogen is not the limiting factor in the system, but rather mass transport of the correct substrate from the bulk to the catalyst surface.

When one increases the substrate concentration under these conditions (see e.g. the test series in examples 14-20 in Table 4), the reaction activity increases for substrate concentrations up to 50 weight %, but upon further increase, a large pressure drop occurs in our reaction system, and we cannot carry out further increases in the substrate concentration. Note that between 40 and 50 weight %, the selectivity decreases dramatically (see S_{Lo} and S_{Ln} for example 18 and 19 in Table 4).

Under these conditions, we use the above-named test series to define and detect the boundary for the substantially homogeneous area in the following way: as long as one observes an increase in the reaction rate and an increase in selectivity upon increasing the amount of substrate from a very low substrate concentration, the system is substantially homogeneous.

FIG. 5 describes the result of flash-point calculations under constant composition and different temperatures and pressures. The single-phase area “Single Phase” is above and to the right of the continuous line. Multiple phases exist under the continuous curves. The area is designated “Multi-Phase” in the figures. The composition of the reaction mixture is given in the box in the upper right-hand side of the figure and is expressed in mol %. One continuous line is calculated for propane and the other for dimethyl ether (DME). DME requires slightly lower pressure than propane to obtain single-phase conditions. The points in the figures are measured limits for propane and the triangles are measured limits for DME. The squares are the critical points for propane and DME.

The substantially homogeneous area is illustrated in the figure with the pale grey area (single-phase area plus a small amount of the multiphase area). The single-phase area at low pressure and high temperature is the traditional gas-phase area. The complex mixtures we deal with lack a clear definition of the critical point. Conventional definitions of supercritical and near-critical conditions are lacking. One can however say that the substantially homogeneous mixture of hydrogen, substrate, and product is in a near-critical or supercritical state.

FIG. 6 describes how one can determine whether one has substantially homogeneous reaction conditions when one has process conditions corresponding to the conditions in FIG. 2 or 3. One observes the reaction rates as a function of substrate concentration. At lower substrate concentrations, the reaction rate increases linearly with substrate concentration. One has single-phase conditions until one reaches the maximal reaction rate. When one further increases the substrate concentration, one reaches a state where the reaction rate begins to decrease, and then falls very sharply. This is because parts of the catalyst surface begin to be covered by drops which comprise a substrate-rich and hydrogen-poor phase. The continuous phase is still rich in hydrogen. Finally, upon further increase in substrate concentration, one reaches a low reaction rate. Here, the catalyst has become completely covered by the substrate-rich and hydrogen-poor phase. The continuous phase around the catalyst has become hydrogen-poor. According to our definition, the reaction conditions are substantially homogeneous as long as the continuous phase is hydrogen-rich.

Figures for Describing Reaction Pathways and Reaction Order for C═C in Fatty Acids.

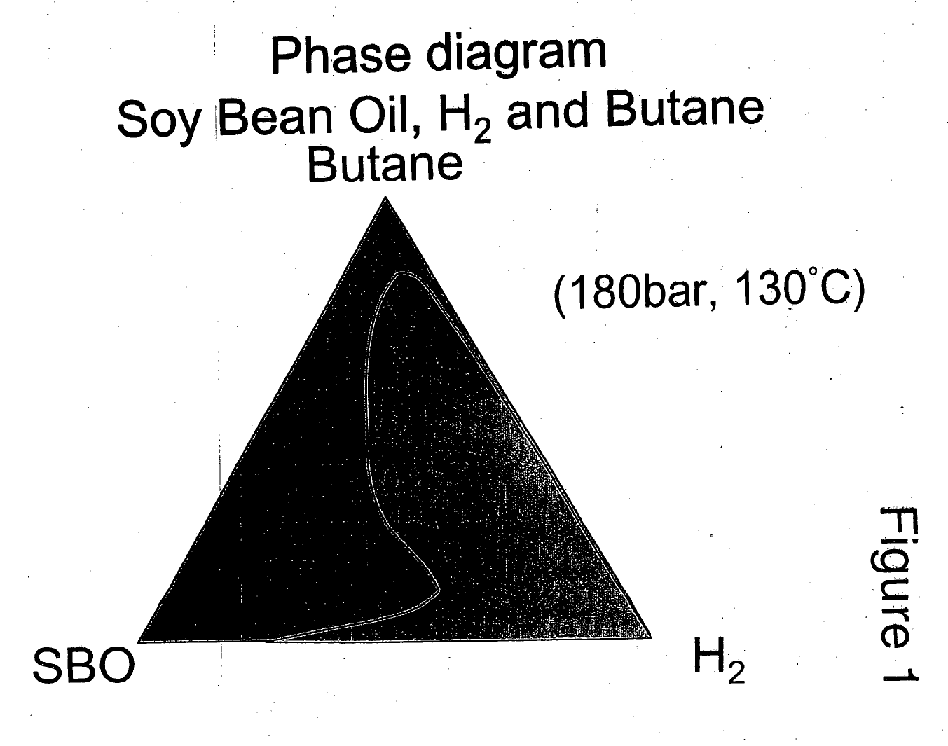

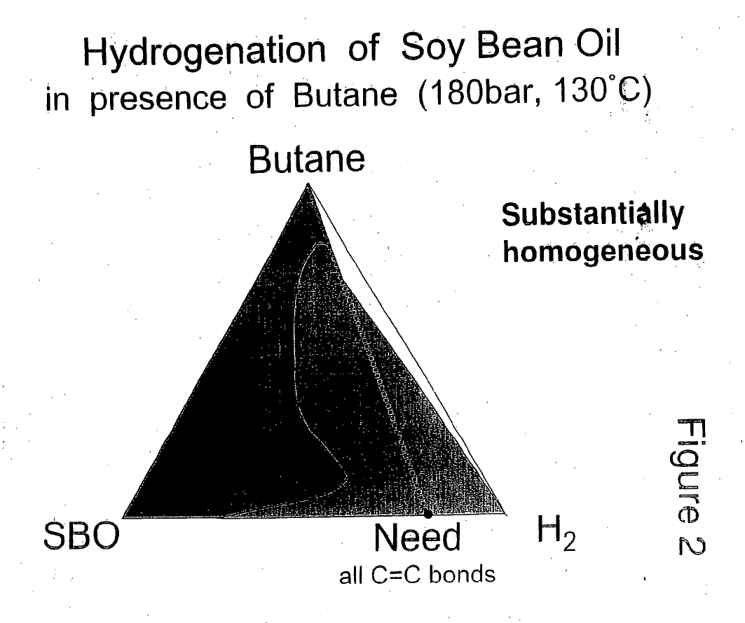

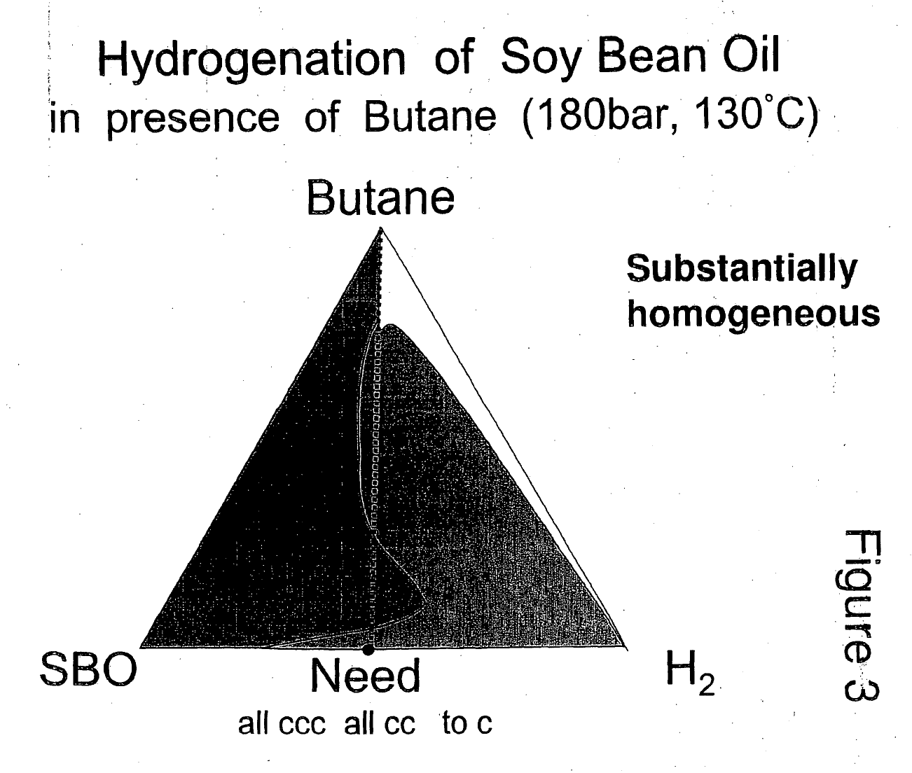

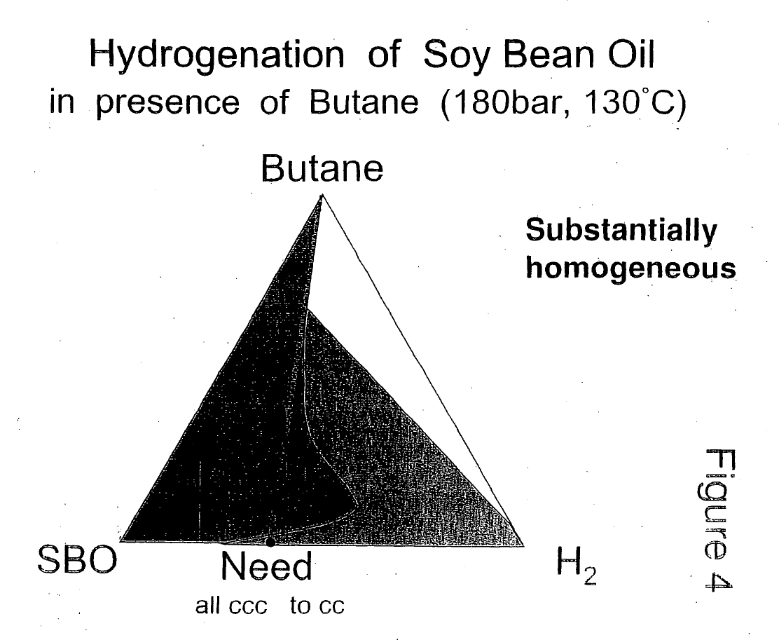

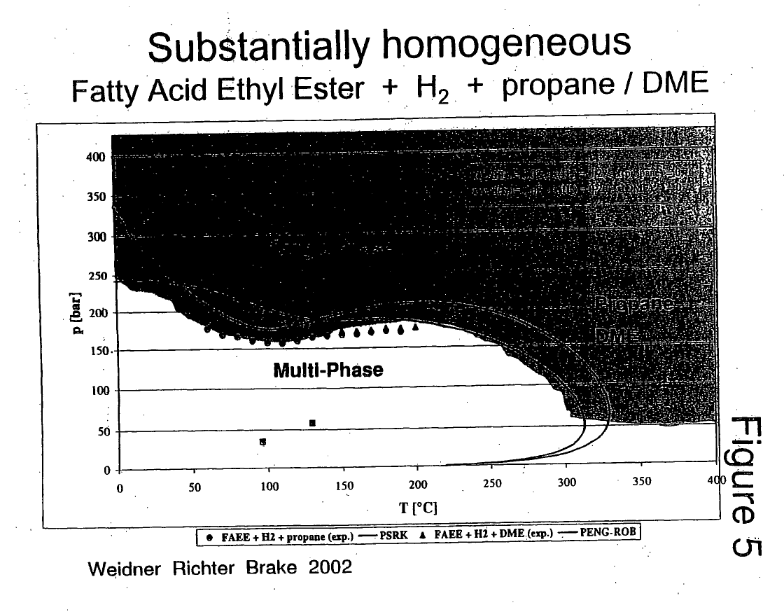

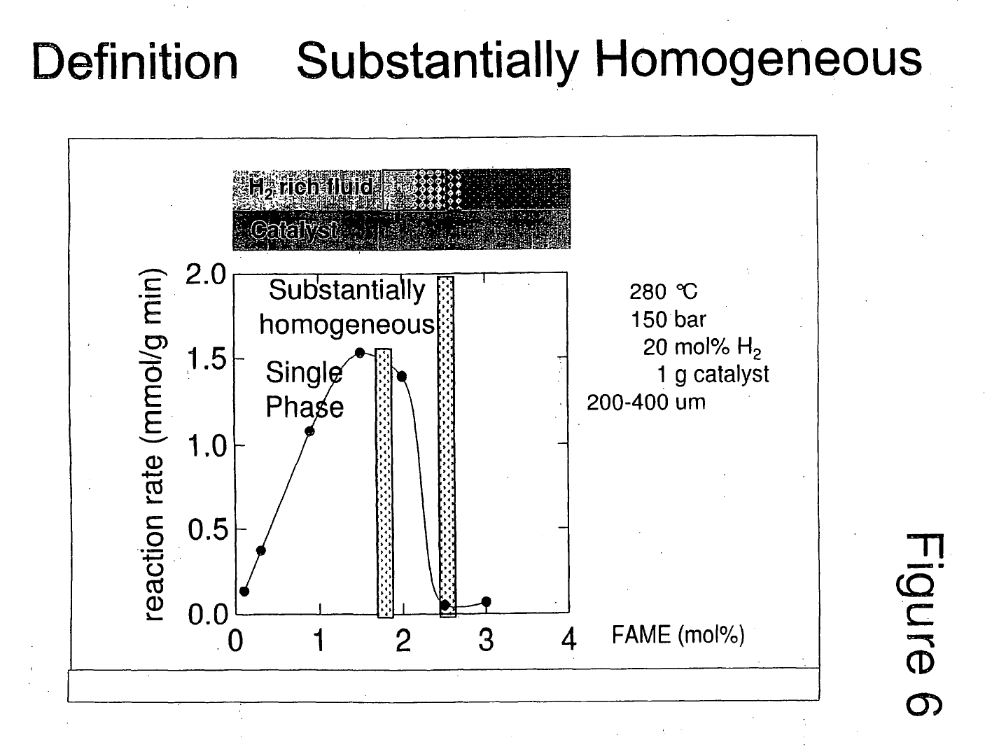

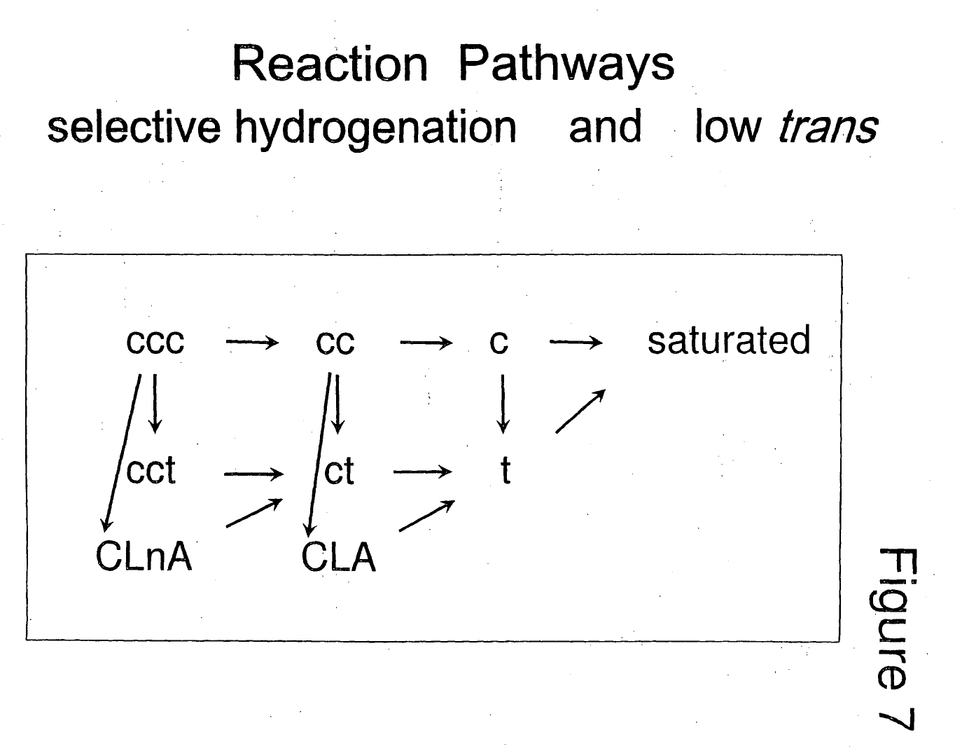

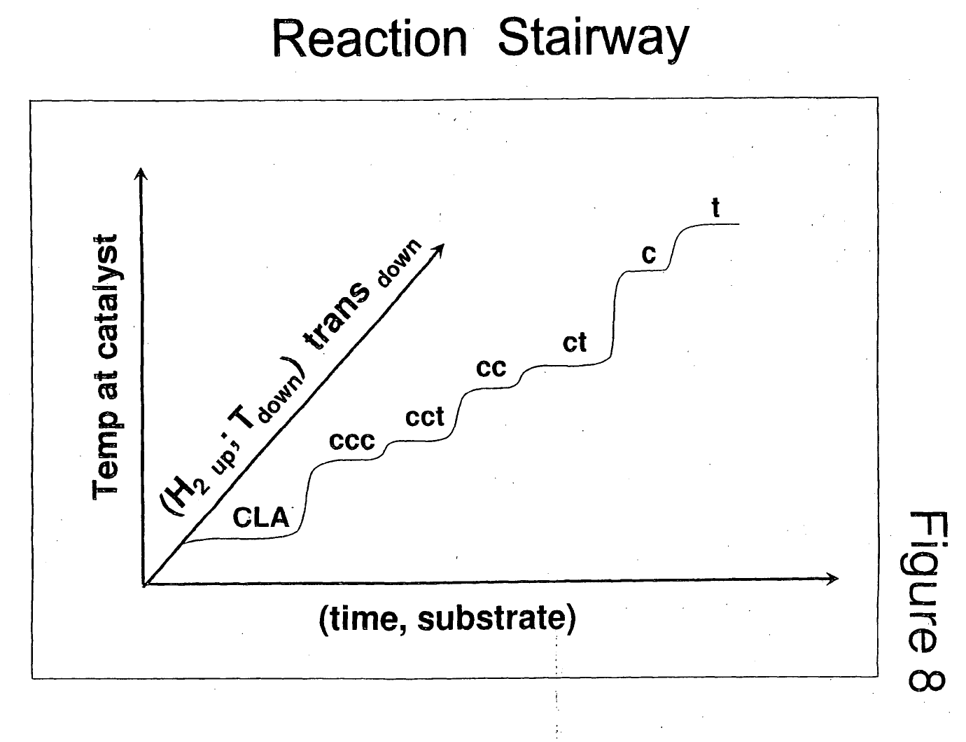

FIG. 7 describes reaction pathways and upon hydrogenation of polyunsaturated fatty acids, ccc, to saturated fatty acids, “saturated”. The figure also describes some reaction pathways to trans-fatty acids, cct, ct and t. In FIGS. 7 and 8, and also in other places in the text, we use the following nomenclature:

c means a cis-bond

t means a trans-bond

In cct and ct, the position of the t is arbitrary. However, the bonds are not conjugated.

CLA means conjugated linoleic acid

CLnA means conjugated linolenic acid

Saturated means saturated fatty acids

FIG. 8 describes to what extent different fatty acids react and how can affect the reaction with various process conditions.

Reaction Paths and Selectivity for C═C in Fatty Acids and Fatty Acid Derivatives

One tends to describe a selectivity as a ratio between two reaction rates. A simple equation for a reaction rate is:
rᵢ=kᵢ exp(Eaᵢ/RT) [H₂]^{a}[C═C]ᵢ^{b}K_{abs} [2]
where r is reaction rate; k is the reaction constant; Eₐ is the activation energy; C═C is a substrate, e.g. a C═C-bond in FIG. 7 and this substrate is described with the index i; a and b are reaction coefficients which tend to be between 0 and 1; K_{abs} is the absorption coefficient of the substrate.

The Y-axis in FIG. 8 describes that the reaction rate is greatest for CLA, and lowest for t. We do not know the values of the reaction rates, but our experiments indicate that their reciprocal relation is described fairly well by FIG. 8. Our experiments indicate however that, for copper at 25° C. the reaction rate constant kᵢ exp(Eaᵢ/RT) K_{abs} for ccc is at least 10 times greater than the same constant for cc (S_{Ln}=9, see Table 4 exp 19). This constant in turn is many hundred times greater than the constant for c (S_{Lo}>100 in many experiments in Table 4).

At low reaction rate (low temperature and a catalyst with low activity) we can control the reaction so that, in the beginning, it is only the ccc-fatty acids which react, mostly to cc, but also to cct. All the c's in ccc have approximately the same reactivity, but the fact that three c's lie near one another makes a ccc-fatty acid in a triglyceride react before a cc fatty acid in the same triglyceride. Only when ccc and cct are finished does the reaction proceed to hydrogenate cc and ct. At high hydrogen pressure, formation of trans is low and isomerisation is practically non-existent. If isomerisation to CLnA should occur, CLnA reacts very quickly to ct. It is important that the temperature increase on the catalyst surface is low, otherwise we do not manage to supply the catalyst with ccc and cc then begins to react.

At a slightly higher reaction rate (slightly higher temperature and same catalyst), we do not manage to supply the catalyst with ccc and cct, and cc then begins to react, mainly to c, but also to ct, which reacts further to t. Isomerisation is very low, but if CLA is produced, it reacts very quickly to t. We have succeeded in completely preventing hydrogenation in this instance, so that neither c nor t has been hydrogenated to saturated. See FIG. 7.

If one further increases the reaction rate (higher temperature and the same catalyst), cc and ct run out on the catalyst surface, and then c also begins to react, mainly to saturated, but also to t. Finally, when c has run out on the catalyst surface, t is hydrogenated to saturated.

We have two reaction coordinates (X-axis) in FIG. 8; one axis for time and substrate, and one axis for hydrogen and temperature.

The hydrogen concentration on the catalyst is crucial in determining the extent of formation of trans-bonds; the higher the amount of hydrogen on the catalyst, the lower the formation of trans-bonds. Lower temperatures reduce the reaction rate and it thereby becomes easier to transport hydrogen to the catalyst surface. We consider a curve for each hydrogen concentration. With increased hydrogen concentration on the catalyst, the relative reaction to cct, ct or t is reduced. The reaction rather goes to cc, c as well as saturated, see FIG. 7.

Increased reaction time and reduced substrate concentration moves the reaction coordinates for time and substrate to the right. In this respect, we consider each type of C═C bond as different substrates.

Selective Hydrogenation of ccc without the Formation of Trans-Fatty Acids.

As shown in FIG. 8, ccc fatty acids are the most reactive fatty acids, and are hydrogenated first. The best selectivity is achieved when the reaction rate is not limited by the transport rate. The transport requirements of ccc fatty acids to the catalyst are therefore very large if one is to obtain high selectivity regarding ccc fatty acids.

The transport rate is controlled by the product of diffusivity and concentration gradient. In this patent, we increase the diffusivity first and foremost by dissolving the oil in a solvent with high diffusivity and low viscosity. By choosing a high concentration of oil, we get a high concentration gradient of ccc fatty acids. However, the concentration should not be chosen to be so high that the product of the diffusivity and concentration gradient falls.

We lower the reactivity in this invention by lowering the temperature and by reducing the catalyst activity (e.g. lower concentration of the active metal and lower surface of the support material). The more active the catalyst, the lower temperature is required for us to obtain good selectivity. A lower hydrogen pressure gives lower activity, but we cannot use this parameter if we want to achieve a low amount of trans. This requires a high hydrogen pressure. The choice of support material and metal affect the selectivity between hydrogenation and formation of trans-fatty acids.

Assume that—for a catalyst—we have found a combination of temperature, hydrogen concentration and substrate concentration and reaction time which gives good selectivity upon hydrogenation of ccc to cc. If we increase the reaction time from these reaction conditions, the turnover of ccc increases until ccc fatty acids run out. To the extent that cct is formed, it is hydrogenated to t. If any CLnA should be formed, this reacts very quickly to ct. The same thing occurs if we reduce the concentration of oil. In both cases, we move to the right on the reaction coordinates in FIG. 8. If the temperature is sufficiently low in the entire reactor, the reaction apparently stops, at this turnover. cc-Fatty acids are hydrogenated further, but with a significantly lower reaction rate than ccc-fatty acids.

Selective Hydrogenation of ccc and cc to c with Minimal Formation of Trans-Fatty Acids.

If we also wish to hydrogenate cc selectively, we control the process so that transport of this component to the catalyst surface does not limit the reaction rate. The mass transport is higher for ccc and cc-fatty acids than for only ccc-fatty acids and the reaction rate is lower for cc-fatty acids. Together, this means that the reactivity can be increased while maintaining high selectivity, i.e. we can raise the temperature with the same catalyst, or we can select a catalyst with higher activity at the same inlet temperature. It is important to monitor the heat which is released upon hydrogenation, particularly at the reactor inlet. If the temperature of the catalyst surface becomes too high, c-fatty acids can begin to react, which results in everything becoming saturated.

If, in an analogous way as above, we assume that we have found a combination of temperature, catalyst, solvent, hydrogen concentration and substrate concentration and reaction time which gives a good selectivity upon hydrogenation of cc to c with a given catalyst. If we increase the reaction time from these reaction conditions, the turnover of cc increases until cc fatty acids run out. To the extent that ct is formed, it is hydrogenated to t. If any CLA should be formed, this reacts very quickly to t. The same thing occurs if we reduce the concentration of oil. In both cases, we move to the right on the reaction coordinates in FIG. 8. If the temperature is sufficiently low in the entire reactor, the reaction apparently stops, at this turnover. c-Fatty acids can be hydrogenated further, but with a significantly lower reaction rate than cc-fatty acids.

Selective Hydrogenation of c-Fatty Acids without Formation of Trans-Fatty Acids.

If we wish to hydrogenate c-fatty acids to saturated fatty acids without hydrogenating trans-fatty acids, we control the process so that the transport of c-fatty acids to the catalyst surface does not limit the reaction rate. The reaction activity can be increased, i.e. we can raise the temperature with the same catalyst, or we can increase the activity of the catalyst at the same inlet temperature, as t-fatty acids have a lower reaction rate than c-fatty acids. However, the temperature on the catalyst surface should not be so high that t-fatty acids begin to react. It is important to monitor the heat which is released upon hydrogenation, particularly at the reactor inlet, so that the temperature of the catalyst surface does not become too high.

If, in an analogous way as above, we assume that we have found a combination of temperature, catalyst, solvent, hydrogen concentration and substrate concentration and reaction time which gives a good selectivity upon hydrogenation of c to saturated with a given catalyst. If we increase the reaction time from these reaction conditions, the turnover of c increases until c-fatty acids run out, while trans-fatty acids remain unhydrogenated. The same thing occurs if we reduce the concentration of oil. In both cases, we move to the right on the reaction coordinates in FIG. 8. If the temperature is sufficiently low in the entire reactor, the reaction apparently stops, at this turnover. t-Fatty acids can be hydrogenated further, but with a significantly lower reaction rate than c-fatty acids.

Hydrogenation of Trans-Fatty Acids to Saturated Fatty Acids.

By increasing the hydrogenation activity, i.e. higher temperature with the same catalyst, or higher activity with approximately the same temperature, it is no problem to hydrogenate t-fatty acids to levels below the level of detection in our processes (IV<0.04).

Substantially Homogeneous

Successful experiments also require that one selects the reaction conditions so that one has a lot of hydrogen on the catalyst, otherwise a lot of trans-bonds are formed.

The substantially homogeneous area which we have defined above comprises a particularly suitable group of reaction mixtures to obtain these conditions

Selection of Process Conditions for Hydrogenation of C═C in Fatty Acids, Triglycerides and Other Fatty Acid Derivatives

Concentrations

A key factor for good selectivity is to provide the catalyst with sufficient amounts of substrate, i.e. C═C of the correct type, and to transport the hydrogenated molecules away sufficiently quickly. A prerequisite for this is that the concentration of the oil should be high, so that the concentration gradient can be high. Another condition is to have a solvent with good transport properties, high diffusivity and low viscosity.

High concentrations of hydrogen on the catalyst are necessary to reduce formation of trans-fatty acids (WO9601304, U.S. Pat. No. 6,265,596, Härröd, Möller). Suitable solvents are the key to obtaining a good result.

For concentration intervals, see tables 1 and 2.

Solvent

A solvent is required which dissolves both oil and hydrogen in high concentrations. Good transport properties are another requirement of the solvent.

Low temperatures lead to triglycerides being hard to dissolve, i.e. a solvent is required which dissolves oil and hydrogen at low temperatures. The solvent should also be allowed in the production of food products.

The technically most interesting solvents for triglycerides are butane and dimethylether (DME). They have good solvating ability for both oil and hydrogen. They further have good transport properties, low viscosity and high diffusivity. Butane is approved for use in food product processes within the EU. DME is not as yet considered as a solvent for food products within the EU.

NH₃ has earlier been shown to be good at reducing the activity of many catalysts, and thereby increasing the selectivity. However, the use of NH₃ in food products is unclear. Therefore, reactions preferably take place in the absence of NH₃ (ammonia) and also in the absence of diamines.

Propane, ethane and CO₂ have a solvating ability for triglycerides which is too low. For other substrates, one can obtain good results with these solvents e.g. FAME (fatty acid methyl esters), propane can work well.

Other solvents which could be considered due to their solvating abilities of the substrate are pentane, hexane, tetrahydrofuran (THF), dioxane, methanol, ethanol and acetone. These have a lower solubility of hydrogen, though, and a lower diffusivity and higher viscosity as compared to e.g. butane or DME. At the low reaction rates which are required for selective hydrogenation of triglycerides, the hydrogen transport requirements are reduced to such an extent that these solvents can be considered. In summary, one can:
normally use CO₂, ethane, propane, butane, pentane, hexane, heptane, tetrahydrofuran, dioxane, dimethylether, methanol, ethanol and acetone and mixtures of these;
preferably use propane, butane, pentane, hexane, heptane, dimethylether, methanol, ethanol and acetone and mixtures of these;
most preferably use propane, butane, dimethylether and ethanol and mixtures of these;
   Total Pressure and Substantially Homogeneous Conditions.

One controls the number of phases in the system with the total pressure. High pressure results in substantially homogeneous conditions in the system, if one has chosen the other process parameters (solvent, concentrations, temperature) suitably. This simplifies mass transport, temperature control and flow patterns in the process. High pressure costs money (maintenance, investments), but reduces the amount of solvent which is required. The optimal balance is a technical-economic balance. For pressure intervals, see table 1.

Catalysts and Hydrogenation Activity

In principle, all catalysts which hydrogenate C═C bonds can be used. In our examples, we have used standard catalysts with Cu and Pd. Other common metals for hydrogenation of C═C are Ni and Pt. In certain circumstances, Co and Ir are used, but other metals and metal combinations exist which can be used.

The processes can be further improved with specially designed catalysts. The following viewpoints can be considered as guidelines for further development. Stable activity is necessary. The catalysts should have a small, specific area, and the particles should be small. The catalytic activity should be low, preferably a low concentration of the active catalyst component. Different types of deliberate inactivation of the catalyst may be interesting.

All of this leads to reduced catalytic activity and ease of diffusion of substrate to, and product from, the catalyst.

The pressure drop across the reactor determines the size of the particles. A guide value is 20-50 μm, yet the proportion and distribution of the smallest particles is very important. The solvent lowers the viscosity of the mixture and this makes it possible to use particles which are significantly smaller than in traditional continuous processes without solvent. Small particles shorten the transport distance in the particle and thereby increase the gradient and mass transport.

It is important to match the activity of the catalyst, so that together with other process parameters (temperature, concentrations, solvents) gives a total hydrogenation activity which agrees with the values provided in Table 1.

Temperatures

By lowering the temperature, we can reduce the catalytic activity more than the mass transport. This increases the selectivity of the reaction. The temperature should therefore be low, preferably room temperature or even lower. The requirement for solubility sets the lower boundary for usable temperatures.

It is important to note that it is the surface temperature of the catalytic site which is the decisive temperature. As a rule, hydrogenation processes are generally strongly exothermic. With substrates such as triglycerides, the most reactive fatty acids (ccc-fatty acids) begin to react. This can easily result in so much heat that cc-fatty acids react, and in the end, even trans-fatty acids. If this happens, there will not be any selective process.

Each catalyst has its own optimal temperature. A more active catalyst requires a lower inlet temperature so that the surface of the catalytic site does not become so high that the next most active bond begins to react and the selectivity is thereby reduced. The relationship between temperature and selectivity is unique for each reaction system. In this patent, we provide general directions and specific levels for the systems which we describe in the examples. One usually ranks the common metals which hydrogenate C═C bonds according to: Cu<Ni<Pt<Pd. The required selectivity is obtained at a higher temperature for copper than nickel, and so on. At the same time, one should be aware that one can reduce the activity of e.g. Pd by reducing the Pd concentration, so that one obtains the same reactivity per unit volume as for a standard Cu catalyst.

With various additives, one can also modify the activity of the catalyst in different ways, e.g. reducing the specific area of the support material. The basic principle still applies: the more active a catalyst is, the lower the optimal temperature will be.

For temperature intervals, see tables 1 and 2.

Reactor Design

Hydrogenation can occur in many different steps in successive reactors. For example, one might selectively hydrogenate bond 1 in a first reactor, bond 2 in a second reactor, and so on.

One may, for instance, hydrogenate all ccc-fatty acids in step one, cc-fatty acids in step two, and so on. This can be particularly valuable if one hydrogenates oils with particularly many polyunsaturated fatty acids, e.g. fish fats. This is because one reduces the transport requirement of hydrogen to the catalyst

Measures which facilitate diffusion of substrate to the catalyst surface are important.

Time

When all other process parameters are fixed, the reaction time is fixed. The reaction rate is minimised, so that mass transport is not the limiting factor. For time intervals, see Table 1.

TABLE 1
Suitable process conditions for hydrogenation of
C═C in fatty acids and fatty acid derivatives

| | | | | Most |
|---|---|---|---|---|
| | | Normally | Preferably | preferably |
| Temperature | ° C. | −50-200 | −10-100 | 0-75 |
| Solvent | weight | 99-5 | 95-20 | 90-50 |
| concentration | % | | | |
| Oil concentration | weight | 2-95 | 5-80 | 10-50 |
| | % | | | |
| Hydrogen | bar | 1-1 000 | 2-500 | 5-300 |
| concentration | | | | |
| Total pressure | bar | 1-1 000 | 10-500 | 20-300 |
| Reaction time | sec. | 1-60 000 | 2-10 000 | 5-3 600 |
| Hydrogenation | mmol | <2.5 | <1.5 | <1 |
| activity | H₂/ls | | | |
| Lowest hydroge- | mmol | 0 | | 0.05 |
| nation activity | H₂/ls | | | |

Product Quality

In this invention, we show how we can obtain unique selectivities when we hydrogenate C═C bonds in fatty acids. We have low reaction rates to trans-fatty acids (S_{II} is low), due to the high amount of hydrogen on the catalyst. We exploit the small differences in activation energies and adsorption coefficients between the different C═C bonds in the fatty acids and obtain unbelievably good selectivities (S_{Ln} och S_{Lo} are very high). The products can be triglycerides, but can also be methyl fatty acids, fatty acids and other fatty acid derivatives.

The selectivity is highest in the beginning of the process, when the substrate concentration is high. We have obtained higher reduction levels and better selectivity than anyone has previously obtained (see Tables 3a, 3b).

TABLE 3a
Product qualities which can be obtained
upon hydrogenation of 18:3

| | | | Most |
|---|---|---|---|
| | Normally | Preferably | preferably |
| Degree of | >85 | <100 | <100 |
| Reduction 18:3 | | | |
| S_{II} | <50 | <40 | <30 |
| S_{Ln} | >1.5 | >3.5 | >4 |
| Exemplified in | Cu 7, 9, 12-13, 15-17 | Cu 12, 13, 16, 17 | Cu 17 |
| | Pd 3 | Pd 4 | Pd 4 |

Degree of Reduction
18:3 = (1 − 18:3end/18:3start)*100
18:3end = 18:3end + 18:cct

TABLE 3b
Product qualities which can be obtained
upon hydrogenation of 18:2+

| | | | Most |
|---|---|---|---|
| | Normally | Preferably | preferably |
| Degree of | >35 | >20 | >20 |
| Reduction 18:2+ | | | |
| S_{II} | <50 | <40 | <30 |
| S_{Lo} | >3.5 | >4 | >8 |
| Exemplified in | Cu 7, 9, 11-13, | Cu 11-13, 15-17 | Cu 11, 13, 17 |
| | 15-17 | Pd 4 | Pd 4 |
| | Pd 3 | FAME 1 | |

Degree of reduction
18:2+ = (1 − (18:2end + 2* 18:3end)/(18:2start + 2* 18:3start))*100
18:2end = 18:2end + 18:ct

Hydrogenation of Other Substrates

The principle can also be used on other substrates in which one has at least two bonds of similar type which can be hydrogenated, but which are of slightly different reaction rates and absorption coefficients and which can be catalysed by the same catalyst. Examples of bonds of similar types are: C═C—C═C; C≡C—C═C; aromatic-C═C; ester-ester; etc.

The principle can furthermore be used on substrates in which one has at least two hydrogenatable bonds of different types but of slightly different reaction rates and absorption coefficients, and which can be catalysed by the same catalyst. Examples of such bonds are C═C—C≡N etc.

The principles for the choice of process parameters for other substrates are the same as discussed above for fatty acids and fatty acid derivatives. In table 2, we have summarised suitable limits for the various process parameters so that one may obtain selective hydrogenations. It is important that one has substantially homogeneous conditions, a lot of hydrogen on the catalyst, a high mass-transport of the substrate to the catalyst and a low catalytic activity chosen in such a way that the mass transport is not the limiting factor.

In terms of solvents, water and ammonia may be usable for polar substrates. In summary, one can:
normally use CO₂, ethane, propane, butane, pentane, hexane, heptane, tetrahydrofuran, dioxane, dimethylether, methanol, ethanol and acetone and mixtures of these;
preferably use propane, butane, pentane, hexane, heptane, dimethylether, methanol, ethanol and acetone and mixtures of these;

most preferably use propane, butane, dimethylether and ethanol and mixtures of these.

TABLE 2
Suitable process conditions for hydrogenation of all substrates

| | | | | Most |
|---|---|---|---|---|
| | | Normally | Preferably | preferably |
| Temperature | ° C. | −50-500 | −10-250 | 0-230 |
| Solvent | weight | 99-5 | 95-20 | 90-50 |
| concentration | % | | | |
| Oil concentration | weight | 1-95 | 5-80 | 10-50 |
| | % | | | |
| Hydrogen | bar | 1-1 000 | 2-500 | 5-300 |
| concentration | | | | |
| Total pressure | bar | 1-1 000 | 5-500 | 10-300 |
| Reaction time | sec. | 1-60 000 | 2-1 000 | 5-1 000 |
| Hydrogenation | mmol | <30 | <3 | <1 |
| activity | H₂/ls | | | |
| Lowest hydroge- | mmol | 0 | | 0.05 |
| nation activity | H₂/ls | | | |

### EXAMPLES

Experimental Methods

The experiments are carried out using the same equipment and in the same way as described in our literature publications (van den Hark et al 1999, Macher 2001, van den Hark 2000).

Hydrogenation is initiated by adding a known amount of hydrogen to a continuous flow of solvent, dimethylether (DME) or butane, and then adding a flow of substrate (rapeseed oil from the local store). The total system pressure was usually 200 bar. The entire reaction mixture is warmed to the desired temperature and passes through a solid catalyst bed which is warmed to the same temperature.

Samples are taken at regular intervals from the reactor outlet for triglyceride analysis by HPLC. Under certain chosen periods, a large amount of produce is collected for methylation and analysis of fatty acid composition by HPLC. Both HPLC methods are based on silver ion chromatography. The triglyceride method is described in Macher, 2001 and Macher Holmqvist 2001 and the methyl ester method is described in van den Hark 2000, and Elfman et al 1997. The method does not analyse chain length. This means that we regard 18:0 as being completely saturated and that this is calibrated against 18:0; 18:t represents all methyl fatty acids with a trans calibrated against 18:t, etc.

A high resolution GC method is used in Tables 8 and 9 to determine the composition of the fatty acids (column: WCOT fused silica 0.25 mm*100 m; gradient [+80° C.->130° C., +45° C./min (0 min)->+220° C., +1° C./min (10 min)]; Injector: 240° C., detector +280° C., carried gas: helium)

Reaction conditions for the individual trials can be found in Tables 4 to 9.

The reaction time is based upon the calculated total volume flow of the reaction mixture (at the current temperature and pressure), divided by the volume of the catalyst bed. The productivity (LHSV) is given as the number of millilitres of substrate which pass through the reaction volume per unit time, i.e. ml_{substrate}/ml_{reactor}*hour. The activity describes the mass transport in the reactor by mmol H₂/litre reactor and second.

Commercial catalysts were used in the experiments

A non-reduced catalyst is used as a Cu catalyst (Engelhard X540, ⅛″ pellets). These pellets were crushed to a powder, and a fraction with a given particle size is used. The particle size was between 90-180 μm. The density was 1.25 kg/l. Activation of the catalyst was carried out with a mixture of nitrogen gas and hydrogen gas (4 to 100 mol-%) which is added to the reactor at the same time as the temperature is raised.

In table 5, a catalyst containing 2 weight % Pd on alumina-silica zeolite (Engelhard) is used as a Pd catalyst. This catalyst is used as delivered, and is warmed to 150° C. in a nitrogen gas-hydrogen gas mixture. The density of the catalyst is 0.5 kg/l

In one experiment (no. 4 in table 5), another catalyst was used. It comprised 1 weight % Pd on carbon powder (Engelhard 5103) and the density is 0.5 kg/l.

In the experiments in tables 8 and 9, a third Pd catalyst from Engelhard (0.1% Pd on alumina, trade code 44451) was used. The density is 1.3 kg/l.

Results and Discussion

Our examples show that we can control the selectivities in a surprisingly good way, although the processes are not optimised.

Copper Table 4

Table 4 describes hydrogenation trials with rapeseed oil, DME and copper catalyst. There are also some selected reference trials which represent the best trials which we found in the literature.

It is well-known that copper can give selective hydrogenation, but that a lot of trans-fatty acids are formed (see introduction of this patent). Our experiments at 100° C. and a short delay period (7-30 sec) (exp. 1,2) give a lot of trans-fatty acids, but also fully-hardened fatty acids. Note that in the reference experiment, (batch, 230° C., 7 h) less saturated has been formed than in our experiments. This shows that it is very difficult to fully harden fatty acids with copper catalysts.

In our experiment at 100° C., the hydrogen requirement corresponds to all ccc-, all cc- and a little c-fatty acids. This requires that “substantially homogeneous” should be interpreted as a hydrogen requirement between FIGS. 2 and 3.

At 50° C. (exp 3-6), the degree of hydrogenation is reduced, and the selectivity improves. The selectivity is comparable to the best data which can be found in the literature, and the amount of trans- is lower (see Sleeter).

In our experiments at 50° C., the hydrogen requirement corresponds to all ccc-, and all cc-fatty acids. This requires that “substantially homogeneous” should be interpreted as a hydrogen requirement as in FIG. 3.

At 25° C. (exp 7-19) the degree of hydrogenation is reduced, and the selectivity improves further (c.f. exp 5 and 9). When we varied the pressure (exp 7-9) there was no apparent effect under the conditions which we investigated.

When we increased the concentration of oil, the degree of hydrogenation was reduced and the selectivity increased dramatically (exp 10-13; exp 14-18). The selectivity reached its maximum at 30-40 weight % oil. In this situation, mass transport is maximal, i.e. the product of the concentration gradient and diffusivity is maximal. At higher concentrations, the diffusivity decreases faster than the gradient increases; i.e. the product of the concentration gradient and diffusivity decreases and mass transport is thereby reduced.

To reach the same degree of hydrogenation at a higher concentration, the delay period is extended (c.f. exp. 13 and exp. 17: both have IV=102, yet exp 13 has 15 weight % and Rt=30, while exp. 17 has 30 weight % and Rt=180).

We can obtain unbelievably good selectivities. The amount of cc-fatty acids does not begin to decrease until ccc-fatty acids fall below 1 weight %. The S_{Ln} is above 9, see exp 18! Saturated fatty acids do not begin to be produced until the amount of cc-fatty acids is less than 5 weight %. S_{Lo} is greater than 100 in the majority of our experiments! It is only when we do not succeed in supplying the catalyst sufficiently with cc-fatty acids that c-fatty acids begin to be hydrogenated to saturated fatty acids.

According to the above reasoning, it is not surprising that the selectivities are very strongly dependent on the amount of each C═C bond. In the tables, we introduce reduction of ccc-fatty acids in column 18:3. Column 18:2+represents reduction of ccc- and cc-fatty acids.

With rapeseed oil as starting material, trans-fatty acids t and ct are produced at approximately the same rate, while IV decreases (see exp 19-16). When the substrate concentration (correct type of C═C bond, not oil concentration) decreases, the reaction rate for production of trans- also falls (see 18:2 and 18:ct in exp 16-14 and in exp 12-10). This reaction rate is low, as there is a lot of hydrogen on the catalyst.

In our experiments at 25° C., the hydrogen requirements correspond to all ccc and a little of cc-fatty acids. This requires that “substantially homogeneous” should be interpreted as a hydrogen requirement between FIGS. 3 and 4.

Palladium Table 5

At 50° C., Pd has the ability to fully hydrogenate triglycerides quickly (ex 1). (c.f. the reaction order described in FIG. 5). In this case, the hydrogen requirements correspond to all ccc-, all cc- and all c-fatty acids. This requires that “substantially homogeneous” should be interpreted as a hydrogen requirement according to FIG. 2.

Small variations in process conditions can give very large variations in the degree of hydrogenation; c.f. ex 1-3. We believe that this results from tristearate falling out in the catalyst, particularly if the concentration of oil is increased.

With a catalyst which is partially inactivated in this way, as in experiment 3, or with a catalyst with activity which is reduced from the very beginning in combination with low temperature, we have succeeded in obtaining excellent results, see exp. 4 Pd 1% and 25° C.

This experiment shows that selectivity primarily depends on the balance between mass transport of the correct C═C bond to the catalyst and the reaction rate. The activity or mass flow in the catalyst is of the same order of magnitude as our copper experiment, and the experiment with good selectivity using palladium (c.f. the activity in Tale 4 and Table 5). It is therefore not the metal per se which is decisive for the selectivity. If one has a very active catalyst and wishes to achieve a selective reaction, one must in some way reduce its reaction rate so that the process is limited by the reaction rate of the catalyst and not by mass transport of the correct C═C bond to the catalyst. Through suitable choice of the catalyst reactivity, suitable solvent, low temperature, suitable concentration of oil and suitable reaction time, we can obtain surprisingly good selectivities between the different C═C bonds in the fatty acids. At the same time, the hydrogen concentration must be high in order to minimise production of trans-fatty acids.

Butane Table 6

Butane works just as well as DME. Butane is approved for food product processes within the EU, while DME is not taken into account. Technically, there is no difference. (c.f. exp 1 and exp 2)

FAME Sunflower Oil Table 7

The example in Table 7 showing hydrogenation of methyl fatty acids shows that we can also obtain very good selectivity for substrates other than triglycerides.

FAEE Hydrogenated and Dehydrogenated Resin Oil, Ethyl Esters Table 8

The examples in Table 8 show that we can hydrogenate cis-bonds very selectively over trans-bonds. At high temperature (110° C.) trans is hydrogenated, but at a lower rate than cis (selectivity=1.6). At low temperature, (80° C.) the selectivity has increased to 51. The activity is very low, less than 0.1 mmol/l s.

CLA Fatty Acids Table 9

The examples in Table 9 show that we can hydrogenate CLA fatty acids to trans fatty acids (t10 and t11) without these fatty acids being hydrogenated any further to saturated fatty acids. The selectivity is infinite (see experiment 3). At high temperatures, significant amounts of c9 are formed: i.e. t11 in CLA is hydrogenated to a certain extent. At low temperature, this bond does not hydrogenate: only t10 and t11 are formed. Experiment 4 shows that a precondition for these selectivities is that we have a lot of hydrogen on the catalyst, as a lot of CLA-isomers are formed and the hydrogen concentration is low in this experiment.

Note that we can carry out selective hydrogenation of CLA at a much higher activity than selective hydrogenation between cis and trans (c.f. activities in Tables 8 and 9).

TABLE 4
Hydrogenation of rapeseed oil in DME and reference data with Cu-catalysts

| | Reaction conditions | | | | | | | | Results | | | | | |
|---|---|---|---|---|---|---|---|---|---|---|---|---|---|---|
| Exp. | | Pₜₒₜ | temp | pH₂ | oil | LHS | Rt | activity | fatty acid compositions wt % | | | | | |
| no | cat. | bar | ° C. | bar | wt % | h⁻¹ | sec | mmolH₂/ls | 18:0 | 18:t | 18:1 | 18:ct | 18:2 | 18:3 |
| | | | | | | | | | 5.4 | | 65.0 | | 22.9 | 6.7 |
| 1 | Cu | 150 | 100 | 30 | 2 | 0.5 | 30 | 0.3 | 31.7 | 50.8 | 12.4 | <1.9 | <1.7 | <1.5 |
| 2 | Cu | 200 | 95 | 30 | 2 | 3.0 | 7 | 0.9 | 10.4 | 40.4 | 44.6 | 1.3 | 2.1 | <1.1 |
| 3 | Cu | 200 | 50 | 30 | 2 | 3.0 | 7 | 0.6 | 6.5 | 8.4 | 72.2 | 2.4 | 9.6 | 0.9 |
| 4 | Cu | 200 | 50 | 40 | 2 | 1.5 | 15 | 0.4 | 5.9 | 14.8 | 71.7 | 0.7 | 6.4 | 0.5 |
| 5 | Cu | 150 | 50 | 30 | 2 | 0.7 | 30 | 0.2 | 6.5 | 15.4 | 72.7 | 1.4 | 3.1 | 0.8 |
| 6 | Cu | 200 | 50 | 10 | 2 | 1.4 | 15 | 0.4 | 4.8 | 22.8 | 68.0 | 1.1 | 2.8 | 0.5 |
| 7 | Cu | 250 | 25 | 40 | 2 | 0.7 | 30 | 0.1 | 5.7 | 8.3 | 76.0 | 2.1 | 7.3 | 0.7 |
| 8 | Cu | 200 | 25 | 40 | 2 | 0.7 | 30 | 0.2 | 4.0 | 15.9 | 74.1 | 0.8 | 4.2 | <1.0 |
| 9 | Cu | 150 | 25 | 40 | 2 | 0.6 | 30 | 0.1 | 6.0 | 6.6 | 75.0 | 2.3 | 9.1 | <1.0 |
| 10 | Cu | 200 | 25 | 40 | 2 | 0.7 | 30 | 0.2 | 4.0 | 15.9 | 74.1 | 0.8 | 4.2 | <1.0 |
| 11 | Cu | 200 | 25 | 40 | 5 | 1.7 | 30 | 0.3 | 5.4 | 2.0 | 75.4 | 1.3 | 14.7 | 1.2 |
| 12 | Cu | 200 | 25 | 40 | 10 | 1.9 | 60 | 0.3 | 5.5 | 3.2 | 73.6 | 1.8 | 15.5 | 0.5 |
| 13 | Cu | 200 | 25 | 40 | 15 | 5.5 | 30 | 0.6 | 3.4 | 1.7 | 73.9 | 1.3 | 18.8 | 0.9 |
| 14 | Cu | 200 | 25 | 40 | 5 | 0.3 | 180 | 0.1 | 7.7 | 13.8 | 69.7 | 1.9 | 5.6 | 1.3 |
| 15 | Cu | 200 | 25 | 40 | 15 | 1.0 | 180 | 0.2 | 5.3 | 6.3 | 76.1 | 1.9 | 9.4 | <1.0 |
| 16 | Cu | 200 | 25 | 40 | 20 | 1.4 | 180 | 0.2 | 4.4 | 2.8 | 76.4 | 2.1 | 13.6 | 0.7 |
| 17 | Cu | 200 | 25 | 40 | 30 | 2.3 | 180 | 0.3 | 4.9 | 1.7 | 71.2 | 1.2 | 20.4 | 0.6 |
| 18 | Cu | 200 | 25 | 40 | 40 | 3.7 | 180 | 0.2 | 3.8 | 1.5 | 68.7 | 1.4 | 22.8 | 1.7 |
| 19 | Cu | 200 | 25 | 40 | 50 | 5.2 | 180 | 0.4 | 6.0 | 0.5 | 69.3 | 0.9 | 18.7 | 4.5 |
| 20 | Cu | 200 | 25 | 40 | 100 | 5.2 | 180 | | not possible to run due to large | | | | | |
| | | | | | | | | | pressure drop in the reactor | | | | | |
| Gritz Göbel DE4103490 | | | | | | | | | 14.2 | — | 23.4 | | 55.8 | 6.6 |
| Henkel 1992 | | | | | | | | | | | | | | |
| Ex 1 | Cu/Zn | | 240 | 20 | 100 | | 7200 | 0.3 | 13.0 | — | 86.0 | | 1.0 | 0.0 |
| Ex V1 | Cu/Mn | | 240 | 20 | 100 | | 21600 | 0.1 | 18.0 | 36.0 | 44.0 | | 2.0 | 0.0 |
| Sleeter WO03059505 | | | | | | | | | 14.2 | | 23.4 | | 55.8 | 6.6 |
| ADM 2003 | | | | | | | | | | | | | | |
| Ex 1 | CuCr | | 230 | 20 | 100 | | 21600 | 0.1 | 16.3 | 38.8 | 30.5 | | 14.4 | 0.0 |

| | Results | | | | | | |
|---|---|---|---|---|---|---|---|
| Exp. | | Selectivity | | | Reduction | | |
| no | IV | S_{II} | S_{Ln} | S_{Lo} | 18:2+ | 18:3 | Comments |
| | 113 | | | | | | Rapeseed oil |
| 1 | 64 | 109 | 0.6 | 6 | 82 | 77 | |
| 2 | 82 | 136 | 0.7 | 39 | 84 | 83 | |
| 3 | 92 | 53 | 2.1 | 62 | 62 | 87 | |
| 4 | 88 | 63 | 1.6 | 264 | 78 | 92 | |
| 5 | 86 | 63 | 1.0 | 159 | 83 | 88 | |
| 6 | 86 | 90 | 1.2 | >500 | 87 | 93 | |
| 7 | 90 | 47 | 1.9 | 361 | 70 | 90 | |
| 8 | 89 | 70 | 1.0 | >500 | 81 | 85 | |
| 9 | 92 | 44 | 1.9 | 122 | 63 | 86 | |
| 10 | 89 | 70 | 1.0 | >500 | 81 | 85 | |
| 11 | 97 | 22 | 2.8 | >500 | 49 | 82 | |
| 12 | 97 | 32 | 4.6 | >500 | 50 | 93 | |
| 13 | 102 | 28 | 5.3 | >500 | 40 | 87 | |
| 14 | 88 | 64 | 1.1 | 48 | 72 | 81 | |
| 15 | 93 | 42 | 1.9 | >500 | 63 | 85 | |
| 16 | 97 | 31 | 3.5 | >500 | 53 | 90 | |
| 17 | 101 | 26 | 7.7 | >500 | 38 | 91 | |
| 18 | 106 | 48 | 9.1 | >500 | 24 | 75 | |
| 19 | 105 | 20 | 1.6 | 29 | 21 | 33 | |
| 20 | not possible to run due to large | | | | | | |
| | pressure drop in the reactor | | | | | | |
| Gritz Göbel DE4103490 | 133 | | | | | | SBO from Kuiper |
| Henkel 1992 | | | | | | | info on trans missing |
| Ex 1 | 76 | — | | | 99 | 100 | |
| Ex V1 | 72 | 59 | 1.8 | 62 | 97 | 100 | |
| Sleeter WO03059505 | 133 | | | | | | SBO from Kuiper |
| ADM 2003 | | | | | | | batch liquid + solid |
| Ex 1 | 84 | 80 | 4.3 | 37 | 79 | 100 | |

< means that the content is so low that we have given it to the lowest amount we have calibrated respective peak in the chromatogram

TABLE 5
Hydrogenation of Rapeseed oil in DME and reference data using Pd-catalysts

| | Reaction conditions | | | | | | | | Results | | | | | |
|---|---|---|---|---|---|---|---|---|---|---|---|---|---|---|
| Exp. | | Pₜₒₜ | temp | pH₂ | oil | LHS | Rt | activity | fatty acid compositions wt % | | | | | |
| no | cat. | bar | ° C. | bar | wt % | h⁻¹ | sec | mmolH₂/ls | 18:0 | 18:t | 18:1 | 18:ct | 18:2 | 18:3 |
| | | | | | | | | | 5.4 | | 65.0 | | 22.9 | 6.7 |
| 1 | Pd | 200 | 50 | 40 | 2 | 7.2 | 2.5 | 7.9 | | | | | | |
| 2 | Pd | 200 | 50 | 20 | 2 | 7.2 | 3.0 | 1.9 | 7.3 | 22.4 | 64.1 | 2.8 | 2.5 | 0.9 |
| 3 | Pd | 200 | 50 | 10 | 2 | 7.2 | 3.0 | 0.9 | 4.8 | 3.6 | 71.9 | 3.2 | 15.6 | <0.9 |
| 4 | Pd | 200 | 25 | 40 | 10 | 2.0 | 23 | 0.18 | 4.4 | 1.7 | 70.8 | 0.8 | 20.8 | 1.6 |
| Heldal JAOCS Vol 66 no 7 | | | | | | | | | 15.1 | | 23.7 | | 53.0 | 8.0 |
| (July 1989) p979-982 | | | | | | | | | | | | | | |
| | Pd | 3 | 80 | 3 | 100 | | 600 | | 18.8 | 1.5 | 25.9 | 1.5 | 47.2 | 5.1 |
| Hsu et al. JAOCS Vol 65 | | | | | | | | | | | | | | |
| no3 (March 1988) p349-356 | | | | | | | | | | | | | | |
| exp 8 | Pd | 50 | 60 | 50 | 100 | | 4500 | 0.4 | | | | | | |
| exp 11 | Pd | 50 | 50 | 50 | 100 | | 1200 | 1.1 | | | | | | |
| Kuiper US4278609 Lever | | | | | | | | | 14.2 | | 23.4 | | 55.8 | 6.6 |
| Brothers 1981 | | | | | | | | | | | | | | |
| Tab 1 | Pd | 1 | 25 | 1 | 25 | | 4860 | 0.03 | 14.5 | 3.0 | 34.3 | 5.0 | 41.8 | 1.4 |
| Tab 3 | Pd | 5 | 25 | 5 | 25 | | 3540 | 0.03 | 14.6 | 2.0 | 29.9 | 4.0 | 47.5 | 2.0 |

| Results | | | | | | | |
|---|---|---|---|---|---|---|---|
| Exp. | | Selectivity | | | Reduction | | |
| no | IV | S_{II} | S_{Ln} | S_{Lo} | 18:2+ | 18:3 | Comments |
| | 113 | | | | | | Rapsolja |
| 1 | 2 | | | | | | only TG analyses |
| 2 | 86 | 94 | 1.0 | 81 | 80 | 86 | |
| 3 | 100 | 52 | 4.5 | >500 | 43 | 87 | |
| 4 | 103 | 27 | 5.1 | >500 | 32 | 77 | |
| Heldal JAOCS Vol 66 no 7 | 133 | best selectivity | | | | | SBO trickel flow |
| (July 1989) p979-982 | | | | | | | |
| | 120 | 24 | 3.2 | 1.0 | 15 | 36 | |
| Hsu et al. JAOCS Vol 65 | 113 | best selectivity | | | | | batch |
| no3 (March 1988) p349-356 | | | | | | | |
| exp 8 | 68 | 23 | 1.0 | 3 | | | Canola |
| exp 11 | 76 | 20 | 1.2 | 2 | | | SBO |
| Kuiper US4278609 Lever | 133 | best selectivity | | | | | SBO batch |
| Brothers 1981 | | | | | | | |
| Tab 1 | 116 | 47 | 5.6 | 28 | 28 | 79 | Acetone + 0.3N NH₃ |
| Tab 3 | 121 | 50 | 7.2 | 11 | 20 | 70 | hexane + 400 mmol NH₃ |

Heldal trans = 3% distribution t ct estimated
Kuiper trans = 8 resp 6% distribution t ct estimated

TABLE 6
Hydrogenation of rapeseed oil using Cu-catalysts and two solvents, DME and butane

| | Reaction conditions | | | | | | | | Results | | | |
|---|---|---|---|---|---|---|---|---|---|---|---|---|
| Exp. | | Pₜₒₜ | temp | pH₂ | oil | LHS | Rt | activity | fatty acid compositions wt % | | | |
| no | cat. | bar | ° C. | bar | wt % | h⁻¹ | sec | mmolH₂/ls | 18:0 | 18:t | 18:1 | 18:ct |
| | | | | | | | | | 5.4 | | 65.0 | |
| 1 | Cu | 200 | 50 | 40 | 2 | 1.6 | 15 | 0.4 | 5.9 | 14.8 | 71.7 | 0.7 |
| 2 | Cu | 200 | 50 | 40 | 2 | 1.3 | 15 | 0.3 | 5.2 | 17.7 | 73.3 | 0.9 |

| | Results | | | | | | | | |
|---|---|---|---|---|---|---|---|---|---|
| | fatty acid | | | | | | | | |
| | compositions | | | | | | | | |
| Exp. | wt % | | | Selectivity | | | Reduction | | |
| no | 18:2 | 18:3 | IV | S_{II} | S_{Ln} | S_{Lo} | 18:2+ | 18:3 | Comments |
| | 22.9 | 6.7 | 113 | | | | | | Rapeseed oil |
| 1 | 6.4 | 0.5 | 88 | 63 | 1.6 | 264 | 78 | 92 | DME |
| 2 | 2.5 | 0.4 | 85 | 68 | 1.1 | >500 | 88 | 93 | butane |

TABLE 7
Hydrogenation of distilled fatty acid methyl esters (FAME) from sunflower oil in DME

| | Reaction conditions | | | | | | | | Results | | | |
|---|---|---|---|---|---|---|---|---|---|---|---|---|
| Exp. | | Pₜₒₜ | temp | pH₂ | oil | LHS | Rt | activity | fatty acid compositions wt % | | | |
| no | cat. | bar | ° C. | bar | wt % | h⁻¹ | sec | mmolH₂/ls | 18:0 | 18:t | 18:1 | 18:ct |
| | | | | | | | | | 7.1 | | 16.6 | |
| 1 | Cu | 200 | 25 | 40 | 20 | 3.1 | 170 | 0.5 | 7.9 | 3.5 | 29.3 | 1.7 |

| | Results | | | | | | | | |
|---|---|---|---|---|---|---|---|---|---|
| | fatty acid | | | | | | | | |
| | compositions | | | | | | | | |
| Exp. | wt % | | | Selectivity | | | Reduction | | |
| no | 18:2 | 18:3 | IV | S_{II} | S_{Ln} | S_{Lo} | 18:2+ | 18:3 | Comments |
| | 76.3 | 0.0 | 145 | | | | | | FAME sunflower oil |
| 1 | 57.6 | 0.0 | 130 | 34 | | 9 | 22 | | |

TABLE 8
Hydrogenation in DME of fatty acid ethyl esters (FAEE)
from hydrogenated and dehydrated castor oil

| | | | | | | | | | Results | | |
|---|---|---|---|---|---|---|---|---|---|---|---|
| | Reaction conditions | | | | | | | | fatty acid compositions wt % | | |
| Exp. | | Pₜₒₜ | temp | pH₂ | oil | LHS | Rt | activity | | C18:1 | |
| no | cat. | bar | ° C. | bar | wt % | h⁻¹ | sec | mmolH₂/ls | C18:0 | c10 | c11 |
| | | | | | | | | | 11.8 | 0.2 | 12.7 |
| 1 | Pd | 200 | 110 | 40 | 10 | 0.7 | 120 | 0.11 | 28.6 | 1.8 | 6.1 |
| 2 | Pd | 200 | 100 | 40 | 10 | 0.7 | 120 | 0.05 | 18.6 | 1.2 | 9.0 |
| 3 | Pd | 200 | 90 | 40 | 10 | 0.7 | 120 | 0.04 | 17.6 | 1.0 | 9.5 |
| 4 | Pd | 200 | 80 | 40 | 10 | 0.6 | 160 | 0.02 | 15.6 | 0.6 | 10.1 |

Results

| | fatty acid compositions wt % | | | | | | | |
|---|---|---|---|---|---|---|---|---|
| Exp. | C18:1 | | | | | | | selectivity |
| no | c12 | c13 | c14 | t10 + t11 | t12 | t13 | IV | def see below |
| | 12.7 | 0.3 | 0 | 30.3 | 30.0 | 0.5 | 75 | |
| 1 | 6.1 | 1.6 | 0.2 | 25.3 | 19.9 | 7.5 | 59 | 1.6 |
| 2 | 9.1 | 0.9 | <0.1 | 28.9 | 26.7 | 3.2 | 68 | 3.7 |
| 3 | 9.6 | 0.8 | <0.1 | 29.3 | 28.0 | 2.3 | 69 | 5.3 |
| 4 | 10.2 | 0.7 | <0.1 | 29.8 | 30.9 | | 71 | 51.0 |

definition selectivity = d(c11 + c12)/d(t10 + t11 + t12 + t13)

TABLE 9
Hydrogenation of conjugated linoleic acids (CLA) in DME

| | | | | | | | | | Results | | | |
|---|---|---|---|---|---|---|---|---|---|---|---|---|
| | Reaction conditions | | | | | | | | fatty acid composition wt % | | | |
| Exp. | | Pₜₒₜ | temp | pH₂ | oil | LHS | Rt | activity | | C18:1 | | |
| no | cat. | bar | ° C. | bar | wt % | h⁻¹ | sec | mmolH₂/ls | C18:0 | c9 | c11 | c12 |
| | | | | | | | | | 0 | 3.7 | <0.1 | 0 |
| 1 | Pd | 200 | 90 | 80 | 10 | 64.2 | 1 | 54.3 | 1.2 | 14.5 | 0.7 | 0.7 |
| 2 | Pd | 200 | 60 | 80 | 10 | 1.2 | 60 | 1.0 | 0.7 | 9.9 | 0.7 | 0.8 |
| 3 | Pd | 200 | 25 | 80 | 10 | 1.2 | 60 | 1.0 | 0 | 5.0 | 0.2 | 0.1 |
| 4 | Pd | 200 | 60 | 0.4 | 10 | 2.3 | 60 | 0.2 | 0.0 | 4.0 | <0.1 | <0.1 |

| Results | |
|---|---|
| fatty acid composition wt % | selectivity |

| | | | | |
|---|---|---|---|---|
| Exp. | C18:1 | CLA | cis | trans |

| | | | | | | | | |
|---|---|---|---|---|---|---|---|---|
| no | t10 | t11 | c9, t11 | t10, c12 | t, t | others | IV | def see below |

| | | | | | | | | | |
|---|---|---|---|---|---|---|---|---|---|
| | 0 | 0 | 89.8 | 3.2 | 1.2 | 1.7 | 168 | | |
| 1 | 40 | 41 | 0 | 0 | 0 | 0 | 83 | 12.7 | 84.5 |
| 2 | 42 | 45 | 0 | 0 | 0 | 0 | 85 | 8.0 | 90.7 |
| 3 | 44 | 51 | 0 | 0 | 0.2 | 0 | 87 | 1.7 | 99.3 |
| 4 | 4 | 3 | 22.4 | 12.8 | 42.2 | 8.4 | 158 | 3.0 | 75.2 |

definition selectivity cis = d (cis)/d (CLA)
definition selectivity trans = d(trans)/d (CLA)

### REFERENCES

Allen R. R., Kiess A. A., 1956, JACOS, 33 (August), 355-359
Allen R. R., 1986, JAOCS, 63(10), 1328-1332
Elfman-Börjesson I., van den Hark S., Härröd M., 1997 JAOCS 74(9)1177-1180
Grau R. J., Cassano A. E., Baltanaás M. A., 1988, Catal. Rev.-Sci. Eng., 30(1), 1-48
van den Hark S., Härröd M., Møller P., 1999, JAOCS. 76(11), 1363-1370.
van den Hark S., PhD Thesis, Chalmers University of Technology, Gothenburg, Sweden,
Heldal J. A., Moulton Sr. K. J., Frankel E. N., 1989, JAOCS, 66 (7), 979-982
Hsu N., Diosady L. L., Rubin L. J., 1989, JACOS, 66(2), 232-236
Hui Y. H., 1996, Bailey's Industrial Oil and Fat Products, (Ed Hui) Wiley, New York, ed 5, vol 4, p 217-
Härröd M., van den Hark S., Macher M.-B., Mℏller P. (2001), In High Pressure Process Technology: Fundamentals and Applications (eds Bertucco and Vetter) Elsevier, p 496-508
Macher M-B., Holmqvist A., J. Sep. Sci, 2001 24(3), 179-185.
Macher M.-B., PhD Thesis, Chalmers University Technology, Gothenburg, Sweden, 2001
Mielke S., 1992, INFORM, 3(6), 695-696
Moulijn J. A., van Leeuwen P. W. N. M., van Santen R. A., 1993, Catalysis: An Integrated Approach to Homogeneous, Heterogeneous and Industrial Catalysis, Elsevier, Amsterdam.
Okkerse C, de Jonge A., Coenen J. W. E., Rozendaal A., 1967, JAOCS, 44, 152-156.
Swern D., 1982, Bailey's Industrial Oil and Fat Products, (Ed Swern) Wiley, New York, ed 4, vol 2
Weidner E., Richter D., Brake C., 2002, Hydrogenation of Fatty Acid Esters in the Presence of Supercritical Fluids—A Thermodynamic Study. In Proceedings Green Solvents for Catalysis, Oct. 13-16, 2002, Bruchsal, Germany

Wahle K. W. J., James W. P. T., 1993, European J. Clinical Nutrition, 47, 828-839

| | |
|---|---|
| GB 670906 | Miyake R., 1952, Nippon Suisan Kabushiki |
| | Kaisha |
| U.S. Pat No. 4278609 | Kuiper J., 1981 Lever Brothers |
| U.S. Pat No. 4307026 | Kuiper J., 1981 Lever Brothers |
| DE 4103490 | Gritz E. Göbel G., 1992, Henkel |
| WO 9601304 | Härröd M., Møller P., 1996 |
| U.S. Pat No. 6265596 | Härröd M., Møller P., 2001 |
| WO 03059505 | Sleeter R., 2003 Archer-Daniels-Midland |

1. Process for hydrogenation of functional groups in hydrogenatable substrates having hydrogenatable functional groups with different reactivities, wherein hydrogen gas is mixed with the substrate and a solvent, the mixture is brought into contact with a catalyst under predetermined conditions of concentration, pressure, temperature and time,
characterized in that
hydrogenation is carried out under process conditions which are adapted to the activity of the catalyst used, wherein the temperature is sufficiently low, the substrate concentration is sufficiently high and the diffusivity is sufficiently high to provide a selective hydrogenation of a functional group having a higher reactivity than one which has a lower reactivity.

2. Process according to claim 1, characterized in that the mixture of hydrogen, substrate and solvent forms a substantially homogeneous phase which is brought into contact with the catalyst.

3. Process according to claim 1, characterized in that the substantially homogeneous mixture of hydrogen, substrate, product and solvent is in a near-critical or critical state.

4. Process according to claim 1, characterised in that the functional group which shows the lower reactivity first begins to be hydrogenated when at least 75%, preferably at least 80% and most preferably at least 90% of the functional group which shows the higher reactivity has been hydrogenated.

5. Process according to claim 1, characterised in that the hydrogenatable functional groups are of the same type but show different reactivities.

6. Process according to claim 1, characterised in that the hydrogenatable functional groups are of the same type but show different, yet similar, reactivities.

7. Process according to claim 5, characterised in that the substrate is a mixture of different molecules and that the hydrogenatable functional groups of the same of different type occur in different molecules.

8. Process according to claim 5, characterised in that the hydrogenatable functional groups of the same or different type occur in the same molecule.

9. Process according to claim 1, characterised in that the hydrogenatable functional groups are C═C groups.

10. Process according to claim 1, characterised in that the hydrogenatable functional groups are lipids, primarily fatty acids and fatty acid derivatives, such as triglycerides and methyl fatty acids.

11. Process according to claim 10, wherein the substrate is fatty acids and/or fatty acid derivatives which are hydrogenated to a degree of reduction of 18:3, i.e. ccc-fatty acids of at least 85%, characterised in that the process conditions are adapted so that the hydrogenated final product comprises an amount of trans-fatty acids in the form of S_{II}-number of highest 50, and a selectivity in the form of S_{Ln}-number of at least 1.5.

12. Process according to claim 1, characterised in that the solvent is selected so that it can dissolve at least 2% w/w, preferably at least 5% w/w and most preferably at least 10% w/w of the substrate at the process conditions in question.

13. Process according to claim 1, characterised in that for lipid substrates, the solvent is selected from the group: carbon dioxide, ethane, propane, butane, pentane, hexane, heptane, tetrahydrofuran (THF), dioxane, dimethylether (DME), methanol, ethanol, acetone and mixtures thereof.

14. Process according to claim 1, characterized in that for lipid substrates, the solvent is selected from the group: propane, butane, pentane, hexane, heptane, dimethylether (DME), ethanol, acetone and mixtures thereof.

15. Process according to claim 1 any of the preceding claims, characterized in that for lipid substrates, the solvent is selected from the group: propane, butane, dimethylether (DME), ethanol, acetone and mixtures thereof.

16. Process according to claim 1, characterized in that for triglyceride substrates, the solvent is selected from the group: butane and dimethylether (DME).

17. Process according to claim 1, characterized in that the reaction is carried out in the absence of ammonia and diamines.

18. Process according to claim 1, characterized in that the concentration of the substrate is at least 2% w/w, preferably at least 5 and most preferably at least 10% w/w.

19. Process according to claim 1, characterized in that the catalyst is a solid-bed catalyst.

20. Process according to claim 1, characterized in that the reaction temperature is at most 200° C., preferably at most 100° C., and most preferably at most 75° C.

21. Process according to claim 1, characterized in that the hydrogen pressure in the substantially homogeneous mixture is at least 1 bar, preferably at least 2 bar and most preferably at least 5 bar.

22. Process according to claim 1, characterized in that the reaction time is at least 1 sec, preferably at least 2 sec and most preferably at least 5 sec.

23. Process according to claim 1, characterized in that, upon hydrogenation of fatty acids and fatty acid derivatives, primarily triglycerides, the hydrogenation activity should be at the most 2.5 mmol H₂/l·s, preferably at most 1.5 mmol H₂/l·s and most preferably at most 1 mmol H₂/l·s.

24. Process according to claim 1, characterized in that, upon hydrogenation of fatty acids and fatty acid derivatives, primarily triglycerides, the hydrogenation activity should be at least 0.05 mmol H₂/l·s.

25. Process according to claim 1, characterized in that the hydrogenation reaction is carried out in sequential steps, so that the majority of a certain type of a selected functional group with a certain reactivity is selectively hydrogenated in each step.

26. Process according to claim 26, characterized in that the different sequential hydrogenation steps are carried out in different reactors with different reaction conditions with regard to one or more factors such as catalyst, temperature or pressure.

27. A partially-hydrogenated product in the form of fatty acids or fatty acid derivatives which have a reduction level of 18:3, i.e. ccc-fatty acids, of at least 85% characterized in that it has a trans-fatty acid content in the form of S_{II}-number of at most 50, and a selectivity in the form of S_{Ln}-number of at least 1.5.

28. A partially-hydrogenated product in the form of fatty acids or fatty acid derivatives which have a reduction level of 18:3, i.e. ccc-fatty acids, under 100%, characterized in that it has a trans-fatty acid content in the form of S_{II}-number of at most 40, preferably at most 30 and a selectivity in the form of S_{Ln}-number of at least 3.5, preferably at least 4.

29. A partially-hydrogenated product in the form of fatty acids or fatty acid derivatives which have a reduction level of 18:2+, i.e. ccc+cc fatty acids, of at least 35%, characterized in that it has a content of trans-fatty acids in the form of S_{II}-number of at most 50, and a selectivity in the form of S_{Lo}-number of at least 3.5.

30. A partially-hydrogenated product according to claim 30, characterized in that it has a reduction level of 18:2+, i.e. ccc+cc fatty acids, of at least 20%, an amount of trans-fatty acids in the form of S_{II}-number of at most 40, preferably at most 30 and a selectivity in the form of S_{Lo}-number of at least 4, preferably at least 8.
